(19) United States
(12) Patent Application Publication
(10) Pub. No.: US 2023/0366887 A1
(43) Pub. Date: Nov. 16, 2023

(54) METHODS OF DETECTING AND USING BIOMARKERS FOR GLYCOGEN STORAGE DISEASES

(71) Applicant: Duke University, Durham, NC (US)

(72) Inventors: Priya Kishnani, Durham, NC (US); Areeg El-Gharbawy, Durham, NC (US)

(73) Assignee: Duke University, Durham, NC (US)

(21) Appl. No.: 18/026,088

(22) PCT Filed: Sep. 21, 2021
(86) PCT No.: PCT/US21/51295

§ 371 Date: Mar. 13, 2023

Related U.S. Application Data
(60) Provisional application No. 63/080,824, filed on Sep. 21, 2020.

(51) Int. Cl. G01N 33/66

(52) U.S. Cl. CPC G01N 33/66; G01N 2800/04; G01N 2800/52

(57) ABSTRACT

There are no FDA-approved disease-modifying therapies for GSDs like GSD Ia and GSD Ib. Disclosed herein are compositions for and methods of detecting GSD biomarkers as well as methods of diagnosing GSD, methods of determining the efficacy of a GSD treatment, methods of monitoring metabolic control status, and methods of determining the efficacy of regulating dietary intake of carbohydrates using the disclosed biomarkers.

### I. CROSS-REFERENCE TO RELATED APPLICATIONS

This application claims the benefit of U.S. Provisional Application No. 63/080,824 filed 21 Sep. 2020, which is incorporated by reference herein in its entirety.

### II. BACKGROUND

Glycogen, a highly branched polymer of glucose molecules, is the body's main storage form of glucose. During the fasted state, glycogen is broken down into its glucose monomers to maintain glucose levels in the blood. Inherited abnormalities in the genes encoding enzymes that enable glycogen synthesis and breakdown are collectively referred to as Glycogen Storage Diseases (GSDs). GSDs are a group of genetic disorders associated with abnormal glycogen metabolism. The group of disorders are generally numbered 0-15 in association with the respective enzymes for glycogen synthesis or breakdown and are identified by affected tissue type (generally liver and/or muscle). Examples of GSDs include, but are not limited to, GSD Type 0 and I-VII, IX, XI, XII, XIII, and XV.

While clinical trials are ongoing, there are currently no FDA-approved disease-modifying therapies for GSDs. Thus, the standard of care remains nutritional intervention.

The present disclosure provides methods of detecting GSD biomarkers, treating a subject having a GSD, determining the efficacy of treatment, monitoring the effect of dietary regulation, methods of monitoring metabolic control status, and more using one or more disclosed GSD biomarkers. These methods can be used alone, or in combination with other treatments such substrate reduction therapy, enzyme replacement therapy, gene therapy, mRNA therapy, or small molecule therapy.

### III. BRIEF SUMMARY

Disclosed herein is a pharmaceutical formulation comprising one or more disclosed vectors, disclosed nucleic acid molecules, and/or disclosed agents in a pharmaceutically acceptable carrier.

Disclosed herein a method of detecting a Glycogen Storage Disease (GSD) biomarker in a subject, the method comprising obtaining a biosample from a subject suspected of having a GSD; obtaining a biosample from a subject not having a GSD; determining the level of one or more cellular metabolites in both biosamples; identifying those cellular metabolites that are differentially present in the biosample obtained from the subject suspected of having a GSD when compared to the biosample from the subject not having a GSD; wherein those differentially present cellular metabolites are biomarkers of a GSD.

Disclosed herein a method of detecting a Glycogen Storage Disease (GSD) biomarker in a subject, the method comprising obtaining a biosample from a subject suspected of having a GSD; determining the level of one or more cellular metabolites in the biosample; identifying those cellular metabolites that are differentially present in the biosample when compared to that of a reference biosample; wherein those differentially present cellular metabolites are biomarkers of a GSD.

Disclosed herein a method of diagnosing a subject with a Glycogen Storage Disease (GSD), the method comprising obtaining a biosample from a subject suspected of having a GSD; determining the level of 1,5-anhydroglucitol (1,5-AG) in the biosample; and when the level of 1,5-AG in the biosample of the subject suspected of having a GSD is significantly higher than that of a reference biosample, then diagnosing the subject with a GSD.

Disclosed herein a method of diagnosing a subject with a Glycogen Storage Disease (GSD), the method comprising obtaining a biosample from a subject suspected of having GSD I; determining the level of 1,5-anhydroglucitol (1,5-AG) in the biosample; and when the level of 1,5-AG in the biosample of the subject suspected of having GSD I is significantly higher than that of a reference biosample, then diagnosing the subject with a GSD Ia or Ib.

Disclosed herein is a method of treating a subject having a Glycogen Storage Disease (GSD), the method comprising obtaining a post-treatment biosample from a subject having a GSD; determining the level of 1,5-anhydroglucitol (1,5-AG) in the biosample; and if the post-treatment level of 1,5-AG represents an improvement over a pre-treatment level of 1,5-AG, or if the post-treatment level of 1,5-AG is within an acceptable range of a reference level, then continuing to administer the treatment.

Disclosed herein is a method of treating a subject having a Glycogen Storage Disease (GSD), the method comprising obtaining a post-treatment biosample from a subject having a GSD; determining the level of 1,5-anhydroglucitol (1,5-AG) in the biosample; and if the post-treatment level of 1,5-AG represents an improvement over a pre-treatment level of 1,5-AG, or if the post-treatment level of 1,5-AG is within an acceptable range of a reference level, then confirming that the treatment is effective.

Disclosed herein is a method of determining the efficacy of a treatment in a subject having a Glycogen Storage Disease (GSD), the method comprising obtaining a pre-treatment biosample from a subject having a GSD and determining the level of 1,5-anhydroglucitol (1,5-AG) in the biosample; administering a treatment; obtaining a post-treatment biosample from the subject and determining the level of 1,5-anhydroglucitol (1,5-AG) in the biosample; and comparing the level of 1,5-AG in the pre-treatment biosample to the level of 1,5-AG in the post-treatment biosample to determine the subject's postprandial glucose excursion (PPGE).

Disclosed herein is a method of determining the effectiveness of a treatment in a subject having a Glycogen Storage Disease (GSD), the method comprising administering a treatment to a subject having a GSD; obtaining a post-treatment biosample from the subject and determining the level of 1,5-anhydroglucitol (1,5-AG) in the biosample; and determining the effectiveness of the treatment by comparing the level of 1,5-AG in the post-treatment biosample to the level of 1,5-AG in either a pre-treatment biosample or a reference biosample.

Disclosed herein is a method of monitoring metabolic control status in a subject having a Glycogen Storage Disease (GSD), the method comprising obtaining a biosample from a subject having a GSD and determining the level of 1,5-anhydroglucitol (1,5-AG) in the biosample; comparing the level of 1,5-AG in the biosample to the level of 1,5-AG in either a biosample previously obtained for the subject or a reference biosample; treating the subject; and repeating the obtaining and comparing steps to determine the effect of the treatment on the metabolic control status.

Disclosed herein is a method of determining the effect of regulating carbohydrate intake in a subject, the method comprising implementing a change in the dietary intake of carbohydrates in a subject having a Glycogen Storage Disease (GSD); obtaining a biosample from the subject; determining the level of 1,5-anhydroglucitol (1,5-AG) in the biosample; and comparing the level of 1,5-AG in the biosample to the level of 1,5-AG in either a pre-dietary change biosample or a reference biosample.

Disclosed herein is a kit comprising one or more disclosed compositions for use in one or more disclosed methods.

### IV. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. A subject can be a newborn baby. In an aspect, a subject can have a glycogen storage disease (GSD), be suspected of having a GSD, or be at risk of developing a GSD. In an aspect, a subject can have a GSD such as, for example, GSD Ia or GSD Ib.

A “patient” refers to a subject afflicted with a GSD. In an aspect, a patient can refer to a subject that has been diagnosed with or is suspected of having a GSD. In an aspect, a patient can refer to a subject that has been diagnosed with or is suspected of having a GSD and is seeking treatment or receiving treatment for a GSD (such as GSD Ia or GSD Ib).

As used herein, the term “diagnosed” means having been subjected to an examination by a person of skill, for example, a physician, and found to have a condition that can be diagnosed or treated by one or more of the disclosed compositions, disclosed methods, or a combination thereof. For example, “diagnosed with a glycogen storage disease” means having been subjected to an examination by a person of skill, for example, a physician, and found to have a condition that can be treated by one or more of the disclosed compositions, disclosed methods, or a combination thereof. For example, “suspected of having a glycogen storage disease” can mean having been subjected to an examination by a person of skill, for example, a physician, and found to have a condition that can likely be treated by one or more of the disclosed compositions, disclosed methods, or a combination thereof. In an aspect, an examination can be physical, can involve various tests (e.g., blood tests, genotyping, biopsies, etc.) and assays (e.g., enzymatic assay), or a combination thereof.

As used herein, the phrase “identified to be in need of treatment for a disorder,” or the like, refers to selection of a subject based upon need for treatment of the GSD. For example, a subject can be identified as having a need for treatment of a GSD like GSD Ia or GSD Ib based upon an earlier diagnosis by a person of skill and thereafter subjected to treatment for the GSD. In an aspect, the identification can be performed by a person different from the person making the diagnosis. In an aspect, the administration can be performed by one who performed the diagnosis.

As used herein, “inhibit,” “inhibiting”, and “inhibition” mean to diminish or decrease an activity, level, response, condition, severity, disease, disease progression, or other biological parameter. This can include, but is not limited to, the complete ablation of the activity, level, response, condition, severity, disease, disease progression, or other biological parameter. This can also include, for example, a 10% inhibition or reduction in the activity, level, response, condition, severity, disease, disease progression, or other biological parameter as compared to the native or control level (e.g., a subject not having a GSD). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a GSD (such as GSD Ia and GSD Ib) can reduce the severity of an established GSD in a subject by 1%-100% as compared to a control (such as, for example, an individual not having a glycogen storage disease). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a GSD. For example, treating a GSD can reduce one or more symptoms of a GSD in a subject by 1%-100% as compared to a control (such as, for example, an individual not having a glycogen storage disease). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established GSD (such as GSD Ia and GSD Ib). It is understood that treatment does not necessarily refer to a cure or complete ablation or eradication of a GSD. However, in an aspect, treatment can refer to a cure or complete ablation or eradication of a GSD.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a GSD is intended. The words “prevent” and “preventing” and “prevention” also refer to prophylactic or preventative measures for protecting or precluding a subject (e.g., an individual) not having a given GSD or GSD-related complication from progressing to that complication (such as, for example, GSD Ia and GSD Ib). In an aspect, preventing a GSD can comprise preventing GSD disease progression.

As used herein, the terms “administering” and “administration” refer to any method of providing one or more of the disclosed compositions, disclosed pharmaceutical formulations, disclosed vectors, disclosed agents, or any combination thereof to a subject. Such methods are well known to those skilled in the art and include, but are not limited to, the following: oral administration, transdermal administration, administration by inhalation, nasal administration, topical administration, intravaginal administration, in utero administration, ophthalmic administration, intraaural administration, otic administration, intracerebral administration, rectal administration, sublingual administration, buccal administration, and parenteral administration, including injectable such as intravenous administration, intra-CSF administration, intracerebroventricular (ICV) administration, intraventricular administration, intra-cisterna magna (ICM) administration, intraparenchymal administration, intrathecal (lumbar, cisternal, or both) administration, intra-lumber administration, intra-arterial administration, intramuscular administration, intra-hepatic administration, and subcutaneous administration. In an aspect, administration can comprise one or more modes of administration, such as, for example, IV administration and intra-CSF administration. In an aspect, any combination of administration can be used such as intra-hepatic administration and IV administration. Administration of a disclosed composition, a disclosed therapeutic agent, a disclosed immune modulator, a disclosed proteasome inhibitor, a disclosed small molecule, a disclosed endonuclease, a disclosed oligonucleotide, and/or a disclosed RNA therapeutic agent can comprise administration directly into the CNS or the PNS. Administration can be continuous or intermittent. Administration of any disclosed composition can comprise a combination of one or more routes of administration.

As used herein, “effective amount” and “amount effective” can refer to an amount that is sufficient to achieve the desired result such as, for example, the treatment and/or prevention of a GSD (e.g., GSD Ia and/or GSD Ib) or a suspected GSD. As used herein, the terms “effective amount” and “amount effective” can refer to an amount that is sufficient to achieve the desired an effect on an undesired condition (e.g., a GSD). For example, a “therapeutically effective amount” refers to an amount that is sufficient to achieve the desired therapeutic result or to have an effect on undesired symptoms but is generally insufficient to cause adverse side effects. In an aspect, “therapeutically effective amount” means an amount of a disclosed isolated nucleic acid molecule, a disclosed vector, or a disclosed pharmaceutical formulation; that (i) treats the particular disease, condition, or disorder (e.g., a GSD), (ii) attenuates, ameliorates, or eliminates one or more symptoms of the particular disease, condition, or disorder e.g., a GSD), or (iii) delays the onset of one or more symptoms of the particular disease, condition, or disorder described herein (e.g., a GSD). The specific therapeutically effective dose level for any particular patient will depend upon a variety of factors including the GSD being treated and the severity of the GSD; the disclosed composition, pharmaceutical formulation, agent or therapeutic agent, immune modulator, proteasome inhibitor, small molecule, endonuclease, oligonucleotide, and/or the RNA therapeutic agent employed; the disclosed methods employed; the age, body weight, general health, sex and diet of the patient; the time of administration; the route of administration; the rate of excretion of the disclosed composition, pharmaceutical formulation, agent or therapeutic agent, immune modulator, proteasome inhibitor, small molecule, endonuclease, oligonucleotide, and/or the RNA therapeutic agent employed; the duration of the treatment; drugs used in combination or coincidental with the disclosed composition, pharmaceutical formulation, agent or therapeutic agent, immune modulator, proteasome inhibitor, small molecule, endonuclease, oligonucleotide, and/or the RNA therapeutic agent employed, and other like factors well known in the medical arts. For example, it is well within the skill of the art to start doses of the disclosed composition, pharmaceutical formulation, agent or therapeutic agent, immune modulator, proteasome inhibitor, small molecule, endonuclease, oligonucleotide, and/or the RNA therapeutic agent at levels lower than those required to achieve the desired therapeutic effect and to gradually increase the dosage until the desired effect is achieved. If desired, then the effective daily dose can be divided into multiple doses for purposes of administration. Consequently, a single dose of the disclosed composition, pharmaceutical formulation, agent or therapeutic agent, immune modulator, proteasome inhibitor, small molecule, endonuclease, oligonucleotide, and/or the RNA therapeutic agent can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, a GSD.

In an aspect, the skilled person can determine an efficacious dose, an efficacious schedule, and an efficacious route of administration for one or more of a disclosed composition, pharmaceutical formulation, agent or therapeutic agent, immune modulator, proteasome inhibitor, small molecule, endonuclease, oligonucleotide, and/or the RNA therapeutic agent to treat or prevent a GSD (such as GSD Ia and/or GSD Ib). In an aspect, the skilled person can also alter, change, or modify an aspect of an administering step to improve efficacy of one or more of a disclosed composition, pharmaceutical formulation, agent or therapeutic agent, immune modulator, proteasome inhibitor, small molecule, endonuclease, oligonucleotide, and/or the RNA therapeutic agent.

As used herein, “modifying the method” can comprise modifying or changing one or more features or aspects of one or more steps of a disclosed method. For example, in an aspect, a method can be altered by changing the amount of one or more of a disclosed composition, pharmaceutical formulation, agent or therapeutic agent, immune modulator, proteasome inhibitor, small molecule, endonuclease, oligonucleotide, and/or the RNA therapeutic agent administered to a subject, or by changing the frequency of administration of one or more of a disclosed composition, pharmaceutical formulation, agent or therapeutic agent, immune modulator, proteasome inhibitor, small molecule, endonuclease, oligonucleotide, and/or the RNA therapeutic agent to a subject, or by changing the duration of time one or more of a composition, pharmaceutical formulation, agent or therapeutic agent, immune modulator, proteasome inhibitor, small molecule, endonuclease, oligonucleotide, and/or the RNA therapeutic agent are administered to a subject.

As used herein, “concurrently” means (1) simultaneously in time, or (2) at different times during the course of a common treatment schedule.

As used herein, “postprandial glucose excursion” or “PPGE” is defined as the change in glucose concentration from before to after a meal, and the incremental glucose area, defined as the area under the glucose curve that is above the premeal (or pre—oral glucose tolerance test [OGTT]) value.

As used herein, a “biomarker” refers to a defined characteristic that is measured as an indicator of normal biological processes, pathogenic processes, or response to an exposure of intervention. In an aspect, a biomarker can be diagnostic (i.e., detects or classifies a pathological condition), prognostic (i.e., predicts the probability of disease occurrence or progression), pharmacodynamic/responsive (i.e., identifies a change in response to a therapeutic intervention), predictive (i.e., predicts how an individual or subject might respond to a particular intervention or event). In an aspect, a biomarker can be diagnostic, prognostic, pharmacodynamic/responsive, and/or predictive at the same time. In an aspect, a biomarker can be diagnostic, prognostic, pharmacodynamic/responsive, and/or predictive at different times (e.g., first a biomarker can be diagnostic and then later, the same biomarker can be prognostic, pharmacodynamic/responsive, and/or predictive). A biomarker can be an objective measure that can be linked to a clinical outcome assessment. A biomarker can be used by the skilled person to make a clinical decision based on its context of use. As used herein, 1,5-AG can be a biomarker.

A “diagnostic biomarker” refers to a biomarker that distinguishes between subjects with a particular disease/ailment/condition and those who do not have the disease/ailment/condition. A “prognostic biomarker” provides information on the likely course of disease/ailment/condition in an individual. A prognostic biomarker can inform the skilled person about the aggressiveness of the disease/ailment/condition and/or the expectation of how a particular subject would fare in the absence of therapeutic intervention. Typically, a prognostic biomarker can identify a patient who is probabilistically at either higher risk for adverse disease-related events or a faster rate of decline in his health status. A “predictive biomarker” is linked to treatment it provides a forecast of the potential for a subject to respond in some identified manner (which may be favorable or unfavorable) to one or more specific treatments. A “response biomarker” is a dynamic assessment that shows a biological response has occurred in a subject after having received a therapeutic intervention.

The term “contacting” as used herein refers to bringing one or more of a disclosed composition, pharmaceutical formulation, agent or therapeutic agent, immune modulator, proteasome inhibitor, small molecule, endonuclease, oligonucleotide, and/or the RNA therapeutic agent together with a target area or intended target area in such a manner that the one or more of a disclosed composition, pharmaceutical formulation, agent or therapeutic agent, immune modulator, proteasome inhibitor, small molecule, endonuclease, oligonucleotide, and/or the RNA therapeutic agent exert an effect on the intended target or targeted area either directly or indirectly. A target area or intended target area can be one or more of a subject's organs (e.g., lungs, heart, liver, kidney, brain, etc.). In an aspect, a target area or intended target area can be any cell or any organ affected by a GSD (such as GSD Ia and/or GSD Ib). In an aspect, a target area or intended target area can be the liver.

As used herein, “determining” can refer to measuring or ascertaining the presence, severity, and/or progression of a GSD, such as, for example, GSD Ia and/or GSD Ib. Methods and techniques used to determine the presence and/or severity of a GSD are typically known to the medical arts. For example, the art is familiar with the ways to identify and/or diagnose the presence, severity, or both of a GSD.

In an aspect, “determining” can refer to measuring or ascertaining the level or amount of one or more cellular metabolites in a biosample. That level or amount can be relative or absolute. Techniques to determine the level or amount or one or more cellular metabolites such as, for example, 1,5-AG, are well known to the skilled technician and include, but are not limited to, mass spectrometry, ultra-performance liquid chromatography (UPLC), high-performance liquid chromatography (HPLC), mass spectrometry in conjunction with UPLC, LC/MS/MS, ELISA, and Western blots.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

In an aspect, a “therapeutic agent” can be a “biologically active agent” or “biologic active agent” or “bioactive agent”, which refers to an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, vaccines, hormones, antibodies (including active antibody fragments sFv, Fv, and Fab fragments), aptamers, peptide mimetics, functional nucleic acids, therapeutic proteins, peptides, or nucleic acids. Other bioactive agents include prodrugs, which are agents that are not biologically active when administered but, upon administration to a subject are converted to bioactive agents through metabolism or some other mechanism. Additionally, any of the compositions of the invention can contain combinations of two or more bioactive agents. It is understood that a biologically active agent can be used in connection with administration to various subjects, for example, to humans (i.e., medical administration) or to animals (i.e., veterinary administration). As used herein, the recitation of a biologically active agent inherently encompasses the pharmaceutically acceptable salts thereof.

As used herein, the term “pharmaceutically active agent” includes a “drug” or a “vaccine” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA) or ribonucleic acid (RNA). Pharmaceutically active agents include the herein disclosed categories and specific examples. It is not intended that the category be limited by the specific examples. Those of ordinary skill in the art will recognize also numerous other compounds that fall within the categories and that are useful according to the invention. Examples include a radiosensitizer, the combination of a radiosensitizer and a chemotherapeutic, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, carbonic anhydrase inhibitors, prostaglandin analogs, a combination of an alpha agonist and a beta blocker, a combination of a carbonic anhydrase inhibitor and a beta blocker, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an anxiolytic agent, a peptidic agent, a biopolymeric agent, an antineoplastic agent, a laxative, an antidiarrheal agent, an antimicrobial agent, an antifungal agent, or a vaccine. In a further aspect, the pharmaceutically active agent can be coumarin, albumin, bromolidine, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, timol hemihydrate, levobunolol hydrochloride, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists (i.e., alpha adrenergic receptor agonist) such as clonidine, brimonidine tartrate, and apraclonidine hydrochloride; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; prostaglandin analogs such as latanoprost, travoprost, and bimatoprost; cholinergics (i.e., acetylcholine receptor agonists) such as pilocarpine hydrochloride and carbachol; glutamate receptor agonists such as the N-methyl D-aspartate receptor agonist memantine; anti-Vascular endothelial growth factor (VEGF) aptamers such as pegaptanib; anti-VEGF antibodies (including but not limited to anti-VEGF-A antibodies) such as ranibizumab and bevacizumab; carbonic anhydrase inhibitors such as methazolamide, brinzolamide, dorzolamide hydrochloride, and acetazolamide; antiarrhythmic agents such as quinidine, lidocaine, tocainide hydrochloride, mexiletine hydrochloride, digoxin, verapamil hydrochloride, propafenone hydrochloride, flecaimide acetate, procainamide hydrochloride, moricizine hydrochloride, and diisopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hydrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides. It is understood that a pharmaceutically active agent can be used in connection with administration to various subjects, for example, to humans (i.e., medical administration) or to animals (i.e., veterinary administration). As used herein, the recitation of a pharmaceutically active agent inherently encompasses the pharmaceutically acceptable salts thereof.

As used herein, a “RNA therapeutic agent” or “RNA therapeutics” can refer to the use of oligonucleotides to target RNA. RNA therapeutics can offer the promise of uniquely targeting the precise nucleic acids involved in a particular disease with greater specificity, improved potency, and decreased toxicity. This could be particularly powerful for genetic diseases where it is most advantageous to aim for the RNA as opposed to the protein. In an aspect, a therapeutic RNA can comprise one or more expression sequences. As known to the art, expression sequences can comprise an RNAi, shRNA, mRNA, non-coding RNA (ncRNA), an antisense such as an antisense RNA, miRNA, morpholino oligonucleotide, peptide-nucleic acid (PNA) or ssDNA (with natural, and modified nucleotides, including but not limited to, LNA, BNA, 2′-O-Me-RNA, 2′-MEO-RNA, 2′-F-RNA), or analog or conjugate thereof. In an aspect, a disclosed therapeutic RNA can comprise one or more long non-coding RNA (lncRNA), such as, for example, a long intergenic non-coding RNA (lincRNA), pre-transcript, pre-miRNA, pre-mRNA, competing endogenous RNA (ceRNA), small nuclear RNA (snRNA), small nucleolar RNA (snoRNA), pseudo-gene, rRNA, or tRNA. In an aspect, ncRNA can be piwi-interacting RNA (piRNA), primary miRNA (pri-miRNA), or premature miRNA (pre-miRNA). In an aspect, a disclosed therapeutic RNA or a RNA therapeutic can comprise antisense oligonucleotides (ASOs) that inhibit mRNA translation, oligonucleotides that function via RNA interference (RNAi) pathway, RNA molecules that behave like enzymes (ribozymes), RNA oligonucleotides that bind to proteins and other cellular molecules, and ASOs that bind to mRNA and form a structure that is recognized by RNase H resulting in cleavage of the mRNA target. Generally speaking, as known to the art, RNAi operates sequence specifically and post-transcriptionally by activating ribonucleases which, along with other enzymes and complexes, coordinately degrade the RNA after the original RNA target has been cut into smaller pieces while antisense oligonucleotides bind to their target nucleic acid via Watson-Crick base pairing, and inhibit or alter gene expression via steric hindrance, splicing alterations, initiation of target degradation, or other events.

The nucleotide sequence for human G6PC is known to the skilled person and the mRNA transcripts can be found at least at DQ033974.1, NM_000151.4, NM_001270397.2, BC136369.1, BC130478.1, or any search for G6PC using for example, GenBank, as a search engine.

The nucleotide sequence for human G6PT is known to the skilled person and the mRNA transcripts can be found at least at NP_001157749.1, NP_001157750.1, NP_001157751.1, NP_001157752.1, NP_001458.1, or any search using G6PT using for example, GenBank, as a search engine.

As used herein, “small molecule” can refer to any organic or inorganic material that is not a polymer. Small molecules exclude large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weight of over 2,000, 3,000, 4,000, 5,000, 6,000, 7,000, 8,000, 9,000, or 10,000). In an aspect, a “small molecule”, for example, can be a drug that can enter cells easily because it has a low molecular weight.

As used herein, a “Z-score” refers to a standard score that is a very useful statistic because it (a) allows one to calculate the probability of a score occurring within the normal distribution and (b) enables one to compare two scores that are from different normal distributions. The standard score does this by converting (in other words, standardizing) scores in a normal distribution to Z-scores in what becomes a standard normal distribution. A Z-score is a measure of how many standard deviations below or above the population mean a raw score is. A Z-score can be placed on a normal distribution curve. Z-scores range from −3 standard deviations (which would fall to the far left of the normal distribution curve) up to +3 standard deviations (which would fall to the far right of the normal distribution curve).

As used herein, “Cohen's D” or “standardized mean difference” refers to one of the most common ways to measure effect size. An effect size is how large an effect is. For example, medication A has a larger effect than medication B. While a p-value can tell you if there is an effect, it won't tell you how large that effect is. Cohen's D specifically measures the effect size of the difference between two means. The formula for Cohen's D (for equally sized groups) is: d=(M1−mM2)/spooled, where M1=mean of group 1, M2=mean of group 2, spooled =pooled standard deviations for the two groups. The formula is: √[(s12+s22)/2].

As used herein, “promoter” or “promoters” are known to the art. Depending on the level and tissue-specific expression desired, a variety of promoter elements can be used. A promoter can be tissue-specific or ubiquitous and can be constitutive or inducible, depending on the pattern of the gene expression desired. A promoter can be native (endogenous) or foreign (exogenous) and can be a natural or a synthetic sequence. By foreign or exogenous, it is intended that the transcriptional initiation region is not found in the wild-type host into which the transcriptional initiation region is introduced.

“Tissue-specific promoters” are known to the art and include, but are not limited to, neuron-specific promoters, muscle-specific promoters, liver-specific promoters, skeletal muscle-specific promoters, and heart-specific promoters.

“Liver-specific promoters” are known to the art and include, but are not limited to, the thyroxin binding globulin (TBG) promoter, the α1-microglobulin/bikunin enhancer/thyroid hormone-binding globulin promoter, the human albumin (hALB) promoter, the thyroid hormone-binding globulin promoter, the α-1-anti-trypsin promoter, the bovine albumin (bAlb) promoter, the murine albumin (mAlb) promoter, the human α1-antitrypsin (hAAT) promoter, the ApoEhAAT promoter comprising the ApoE enhancer and the hAAT promoter, the transthyretin (TTR) promoter, the liver fatty acid binding protein promoter, the hepatitis B virus (HBV) promoter, the DC172 promoter comprising the hAAT promoter and the α1-microglobulin enhancer, the DC190 promoter comprising the human albumin promoter and the prothrombin enhancer, or any other natural or synthetic liver-specific promoter. In an aspect, a liver specific promoter can comprise about 845-bp and comprise the thyroid hormone-binding globulin promoter sequences (2382 to 13), two copies of α1-microglobulinybikunin enhancer sequences (22,804 through 22,704), and a 71-bp leader sequence as described by Ill CR, et al. (1997).

As used herein, an “inducible promoter” refers to a promoter that can be regulated by positive or negative control. Factors that can regulate an inducible promoter include, but are not limited to, chemical agents (e.g., the metallothionein promoter or a hormone inducible promoter), temperature, and light.

As used herein, the term “immunotolerant” refers to unresponsiveness to an antigen (e.g., a vector, a therapeutic protein, a transgene product, etc.). An immunotolerant promoter can reduce, ameliorate, or prevent transgene-induced immune responses that can be associated with gene therapy. Assays known in the art to measure immune responses, such as immunohistochemical detection of cytotoxic T cell responses, can be used to determine whether one or more promoters can confer immunotolerant properties.

As used herein, “codon optimization” can refer to a process of modifying a nucleic acid sequence for enhanced expression in the host cells of interest by replacing one or more codons or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.” Many methods and software tools for codon optimization have been reported previously. (See, for example, genomes.urv.es/OPTIMIZER/).

As used herein, “substrate reduction therapy” or “SRT” refers to methods of reducing the level of the substrate to a point where residual degradative activity of one or more enzymes is sufficient to prevent substrate accumulation. Generally, SRT aims to use small molecule inhibitors of biosynthesis to reduce the concentration of accumulating substrate to a level where the residual degradative enzymes can maintain homeostasis. In an aspect, SRT can be used to reduce activity and/or expression level of one or more other enzymes in glycogen synthesis and/or glycogen breakdown and/or glucose breakdown. In an aspect, SRT can comprise siRNA-based therapies, shRNA-based therapies, antisense therapies, gene-editing therapies, and therapies using one or more small molecules or peptide drugs. In an aspect, SRT can comprise administration of one or more small molecules that can traverse the blood-brain barrier in quantities that are therapeutic for a subject having neuropathic glycogen storage disease. In an aspect, SRT can comprise administration of one or more small molecules that do not traverse the blood-brain barrier in quantities but are nonetheless therapeutic for a subject having neuropathic GSD. In an aspect, a disclosed small molecule used in SRT can be orally delivered.

As used herein, “immune tolerance,” “immunological tolerance,” and “immunotolerance” refers to a state of unresponsiveness or blunted response of the immune system to substances (e.g., a disclosed isolated nucleic acid molecule, a disclosed vector, a disclosed transgene product, a disclosed pharmaceutical formulation, a disclosed therapeutic agent, etc.) that have the capacity to elicit an immune response in a subject. Immune tolerance is induced by prior exposure to a specific antigen. Immune tolerance can be determined in a subject by measuring antibodies against a particular antigen or by liver-restricted transgene expression with an AAV vector. Low or absent antibody titers over time is an indicator of immune tolerance. For example, in some embodiments, immune tolerance can be established by having IgG antibody titers of less than or equal to about 12,000, 11,500, 11,000, 10,500, 10,000, 9,500, 9,000, 8,500, 8,000, 7,500, 7,000, 6,500, or 6,000 within following gene therapy (such as the administration of the transgene encoding, for example, one or more proteins and/or enzymes involved in glycogen synthesis and/or glycogen breakdown and/or glucose breakdown) or a CpG-free and codon optimized ORF for one or more of these proteins and/or enzymes.

In an aspect, also disclosed herein are partial self-complementary parvovirus (e.g., a disclosed AAV) genomes, plasmid vectors encoding the parvovirus genomes, and parvovirus (e.g., a disclosed AAV) particles including such genomes. In an aspect, provided herein is a plasmid vector comprising a nucleotide sequence encoding a disclosed parvovirus genome such as for example, a disclosed AAV. In an aspect, provided herein is a partial self-complementary parvovirus genome including a payload construct, parvovirus ITRs flanking the payload construct, and a self-complementary region flanking one of the ITRs. A self-complementary region can comprise a nucleotide sequence that is complementary to the payload construct. A disclosed self-complementary region can have a length that is less the entire length of the payload construct.

In an aspect, a disclosed self-complementary region of a disclosed parvovirus genome can comprise a minimum length, while still having a length that is less the entire length of the payload construct. In an aspect, a disclosed self-complementary region can comprise at least 50 bases in length, at least 100 bases in length, at least 200 in length, at least 300 bases in length, at least 400 bases in length, at least 500 bases in length, at least 600 bases in length, at least 700 bases in length, at least 800 bases in length, at least 900 bases in length, or at least 1,000 bases in length.

As used herein, “immune-modulating” refers to the ability of a disclosed isolated nucleic acid molecules, a disclosed vector, a disclosed pharmaceutical formulation, or a disclosed agent to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors, including lymphokines and antibodies, that have autocrine, paracrine, and endocrine effects on immune cells.

As used herein, “immune modulator” refers to an agent that is capable of adjusting a given immune response to a desired level (e.g. as in immunopotentiation, immunosuppression, or induction of immunologic tolerance). Examples of immune modulators include but are not limited to aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGF1R antibodies, an IL2 mutein, a BTK inhibitor, or a combination thereof. In an aspect, a disclosed immune modulator can comprise one or more Treg (regulatory T cells) infusions (e.g., antigen specific Treg cells to AAV). In an aspect, a disclosed immune modulator can be bortezomib or SVP-Rapamycin. In an aspect, an immune modulator can be administered by any suitable route of administration including, but not limited to, in utero, intra-CSF, intrathecally, intravenously, subcutaneously, transdermally, intradermally, intramuscularly, orally, transcutaneously, intraperitoneally (IP), or intravaginally. In an aspect, a disclosed immune modulator can be administered using a combination of routes. Administration can also include hepatic intra-arterial administration or administration through the hepatic portal vein (HPV). Administration of an immune modulator can be continuous or intermittent, and administration can comprise a combination of one or more routes.

In an aspect, a disclosed immune modulator such as methotrexate can be administered at a transient low to high-dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., a disclosed composition, pharmaceutical formulation, agent or therapeutic agent, immune modulator, proteasome inhibitor, small molecule, endonuclease, oligonucleotide, and/or the RNA therapeutic agent) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy to a subject having or diagnosed with a GSD (such as GSD Ia and/or GSD Ib).

Disclosed are the components to be used to prepare the disclosed isolated nucleic acid molecules, disclosed vectors, or disclosed pharmaceutical formulations as well as the disclosed isolated nucleic acid molecules, disclosed vectors, or disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. Compositions for Use in the Disclosed Methods

Disclosed herein is a pharmaceutical formulation comprising one or more disclosed vectors, disclosed nucleic acid molecules, and/or disclosed agents in a pharmaceutically acceptable carrier. In an aspect, a disclosed pharmaceutical formulation can comprise one or more agents that modulate the expression level of one or more disclosed differentially present cellular metabolites such as, for example, increasing or decreasing the level of the cellular metabolite (e.g., 1,5-AG). In an aspect, a disclosed pharmaceutical formulation can comprise one or more agents that modulate the level of one or more differentially present cellular metabolites. In an aspect, a disclosed pharmaceutical formulation can comprise one or more agents that target one or more differentially present cellular metabolites associated with impaired glycogenolysis, impaired gluconeogenesis, impaired ketogenesis, lactic acidosis, hyperuricemia, hyperlipidemia, or any combination thereof. In an aspect, a disclosed pharmaceutical formulation can comprise one or more agents that modulate the expression and/or activity level of G6Pase and/or G6PT. In an aspect, a disclosed agent can target G6PT dysfunction or G6PT deficiency. In an aspect, a disclosed agent can target renal sodium glucose co-transporter 2 (SGLT2), such as, for example, empagliflozin, canagliflozin, dapagliflozin, ipragliflozin, luseogliflozin, and Tofogliflozin. In an aspect, the SGLT2 inhibitor can be empagliflozin. In an aspect, a disclosed pharmaceutical formulation can comprise an isolated nucleic acid molecule encoding a protein that is deficient and/or impaired in the subject such as, for example, a deficient and/or impaired protein having a role in glycogenolysis and/or gluconeogenesis. In an aspect, a disclosed pharmaceutical formulation can comprise an isolated nucleic acid molecule encoding a native or recombinant protein, such as, for example, alpha-glucosidase (GAA), α-galactosidase A (GLA), acid β-glucosidase, α-L-iduronidase, iduronate 2-sulfatase, lysosomal acid lipase, liver glycogen synthase, muscle glycogen synthase, glucose-6-phosphatase α, glucose-6-phosphate transporter, α-glucosidase, glycogen debranching enzyme, glycogen branching enzyme, muscle glycogen phosphorylase, liver glycogen phosphorylase, muscle phosphofructose kinase, phosphorylase kinase (α2 subunit), phosphorylase kinase (β subunit), phosphorylase kinase (γ subunit), phosphorylase kinase (α1 subunit), muscle phosphoglycerate mutase, glucose transporter 2, aldolase A, γ2-subunit of AMP-activated protein kinase (AMPK), β-enolase, glycogenin-1, dystrophin, factor VIII, lysosomal-associated membrane protein 2, laforin, or malin, or a fragment thereof. In an aspect, a disclosed isolated nucleic acid can comprise the sequence for AGA, AGL, ALSOA, ARSA, ARSB, ASAH1, ATG5, ATG7, CGI58, CLCN5, CLN3, CLN5, CLN6, CLN8, CTNS, CTSA, CTSK, ENO3, EPM2A, FIG4, FUCA1, G6PC, G6PT (i.e., G6PT1, G6PT2, G6PT3), GAA, GALC, GALNS, GBA, GDE, GEB1, GLA, GLB1, GM2A, GNPTAB, GNPTG, GNS, GUSB, GYG1, GYS1, GYS2, HEXA, HEXB, HGSNAT, HYAL1, IDS, IDUA, LAMP2, LIPA, MAN2B1, MANBA, MCOLN1, mTORC1, NAGA, NAGLU, NEU1, NHLRC1, NPC1, NPC1, NPC2, OCRL, PNPLA2, PPT1, PSAP, PFKM, PHKA2, PHK, PHKG2, PHKA1, PGAM2, PRKAG2, SGSH, SLC17A5, SLC37A4, SLC38A9, SLC9A6, SMPD1, SUMF1, and TPP1.

### C. Methods of Detecting a Glycogen Storage Disease (GSD)

Disclosed herein is a method of detecting a Glycogen Storage Disease (GSD) biomarker in a subject, the method comprising obtaining a biosample from a subject suspected of having a GSD; obtaining a biosample from a subject not having a GSD; determining the level of one or more cellular metabolites in both biosamples; identifying those cellular metabolites that are differentially present in the biosample obtained from the subject suspected of having a GSD when compared to the biosample from the subject not having a GSD; wherein those differentially present cellular metabolites are biomarkers of a GSD.

In an aspect, a biosample can comprise EDTA, blood, serum, or plasma. In an aspect, determining the level of the one or more cellular metabolites in a biosample can comprise using mass spectrometry, ultra-performance liquid chromatography (UPLC), high-performance liquid chromatography (HPLC), mass spectrometry in conjunction with UPLC, LC/MS/MS, ELISA, Western blots, or any combination thereof. The skilled person can identify the method or technique that is most suitable for a given biosample. In an aspect, the same method or technique can be used for one or more biosamples or for all biosamples. In an aspect, a different method or technique can be used for one or more biosamples or for all biosamples.

In an aspect, a subject suspected of having a GSD can be an adult, a young adult, an adolescent, a child, or a newborn infant. In an aspect, a subject suspected of having a GSD can be a newborn infant. In an aspect, a subject can be a male or a female.

In an aspect, the one or more differentially present cellular metabolites can be associated with impaired glycogenolysis, impaired gluconeogenesis, impaired ketogenesis, or any combination thereof. In an aspect, the one or more differentially present cellular metabolites can be associated with lactic acidosis, hyperuricemia, hyperlipidemia, or any combination thereof. In an aspect, the one or more differentially present cellular metabolites can be associated with impaired glycogenolysis, impaired gluconeogenesis, impaired ketogenesis, lactic acidosis, hyperuricemia, hyperlipidemia, or any combination thereof.

In an aspect, the one or more cellular metabolites can comprise 1,5-anhydroglucitol (1,5 AG), 25-hydroxyvitamin D, albumin, adiponectin, analytes associated with CLIX score, analytes associated with HOMA (Homeostasis Model Assessment) score, analytes associated with IR score, anti-islet cell cytoplasmic (anti-ICA) auto-antibodies, C-peptide, cystatin C, D-mannose, estimated glomerular filtration rate (eGFR), F2-isoprostanes, ferritin, fibrinogen, free fatty acids, fructosamine, gamma-glutamic transferase (GGT), glucose, glutamic acid decarboxylase (anti-GAD) auto-antibodies, glycation gap (glycosylation gap), HDL, HDL-c, hemoglobin (Hb) A1c, homocysteine, insulin, intact pro-insulin, LDL, LDL-c, leptin, leptin/adiponectin ratio, linoleoyl glycerophosphocholine (L-GPC), lipoprotein-associated phospholipase A2 (Lp-PLA2), mannose binding lectin (MBL) activity, mannose, mannose-binding lectin (MBL) amount, myeloperoxidase, oleic acid (OA), pro-insulin, serum amylase, serum cholinesterase, serum creatinine, triglycerides, TSH, uric acid, vitamin B12, and any combination thereof. In an aspect, one or more of the cellular metabolites can be differentially present. In an aspect, a combination of cellular metabolites can be differentially present.

In an aspect, the one or more cellular metabolites can comprise 1,5-AG, glucose, uric acid, lactate, triglycerides, and any combination thereof. In an aspect, the differentially present cellular metabolite can comprise 1,5-AG. In an aspect, the one or more cellular metabolites can comprise 1,5-AG, serum cholinesterase, albumin, and any combination thereof.

In an aspect, a subject suspected of having a GSD can have a higher level of 1,5-AG than does a subject not having a GSD. In an aspect, the higher level of 1,5-AG in a subject suspected of having a GSD can be associated with a Z-score of at least +1.5, at least +2, or more than +2. In an aspect, the differential level of 1,5-AG can comprise at least a Cohen's d effect size of at least 0.2, at least 0.5, at least 0.8, or greater than 0.8. In an aspect, the differential level of 1,5-AG can comprise at least a Cohen's d effect size of at least 1, at least 2, at least 3, or greater than 3.

In an aspect, one or more differentially present cellular metabolites can be associated with a Z-score of at least +1.5, at least +2, or more than +2, or one or more differentially present cellular metabolites can be associated with a Z-score of at least −1.5, at least −2, or more than −2. In an aspect, the differential level of one or more cellular metabolites can comprise at least a Cohen's d effect size of at least 0.2, at least 0.5, at least 0.8, or greater than 0.8. In an aspect, the differential level of one or more cellular metabolites can comprise at least a Cohen's d effect size of at least 1, at least 2, at least 3, or greater than 3.

In an aspect, a disclosed method can comprise diagnosing a subject suspected of having a GSD with having a GSD. In an aspect, a disclosed subject can have GSD I, GSD II, GSD III, GSD IV, GSD V, GSD VI, GSD VII, GSD IX, GSD XI, GSD XII, GSD XIII, GSD IX, GSD XIV, or GSD XV. In an aspect, a disclosed subject can have GSD III, GSD VI, GSD IX, or Fanconi-Bickel syndrome. In an aspect, a disclosed subject can have GSD Ia or GSD Ib.

In an aspect, disclosed method can comprise treating the subject diagnosed with having a GSD. In an aspect, treating the subject can comprise administering to the subject one or more agents that modulate the level of one or more differentially present cellular metabolites. In an aspect, a disclosed agent can target G6PT dysfunction of G6PT deficiency. In an aspect, treating the subject cam comprise administering to the subject one or more agents that target renal sodium glucose co-transporter 2 (SGLT2), such as, for example, empagliflozin, canagliflozin, dapagliflozin, ipragliflozin, luseogliflozin, and Tofogliflozin. In an aspect, the SGLT2 inhibitor can be empagliflozin.

In an aspect of a disclosed method, treating a subject can comprise administering to the subject to the subject ERT, gene therapy, mRNA therapy, small molecule therapy, SRT, or any combination thereof. In an aspect of a disclosed method, treating a subject can comprise administering to the subject a combination of one or more agents with ERT, gene therapy, mRNA therapy, small molecule therapy, SRT, or any combination thereof.

In an aspect of a disclosed method, treating a subject can comprise implementing a change in the subject's dietary intake of carbohydrates. In an aspect, a change in a subject's dietary intake of carbohydrates can comprise adding carbohydrates to the subject's diet, or removing carbohydrates from the subject's diet, or changing the type of carbohydrates in the subject's diet, or changing the frequency of carbohydrates consumed by the subject.

In an aspect, a subject's treatment can comprise ingesting one or more anaplerotic agents (e.g., medium-even-chain triglycerides (MCT), triheptanoin, etc.). In an aspect, the one or more anaplerotic agents can reduce the amount of cornstarch and/or carbohydrates required by the subject to, for example, maintain metabolic control.

In an aspect, implementing a change in the dietary intake of carbohydrates can comprise adding carbohydrates to the subject's diet, or removing carbohydrates from the subject's diet, or changing the type of carbohydrates in the subject's diet, or changing the frequency of carbohydrates consumed by the subject. In an aspect, the one or more anaplerotic agents can reduce the amount of carbohydrates required to achieve metabolic control in the subject.

In an aspect of a disclosed method, treating a subject can comprise administering cornstarch to the subject. Cornstarch is known to the art and can comprise uncooked cornstarch, modified cornstarch, a cornstarch derivative, a cornstarch alternative, or any combination thereof. In an aspect, cornstarch can comprise Glycosade . In an aspect of a disclosed method, treating a subject can comprise administering glycoside to the subject.

In an aspect, any step in the disclosed method can be repeated one or more times. In an aspect, one or more steps in the disclosed method can be repeated one or more times.

In an aspect, a step of a disclosed method can be modified. For example, in an aspect, modifying the method can comprise modifying or changing one or more features or aspects of one or more steps of a disclosed method. For example, in an aspect, a disclosed method can be altered by changing the amount of one or more of the disclosed agents, by changing the route or frequency of administration of one or more of the disclosed agents, or by changing the duration of time one or more of the disclosed agents are administered to a subject.

In an aspect of a disclosed method, gene therapy can comprise administering to the subject an isolated nucleic acid molecule encoding a protein that is deficient and/or impaired in the subject such as, for example, a deficient and/or impaired protein having a role in glycogenolysis and/or gluconeogenesis. In an aspect, a disclosed isolated nucleic acid can comprise the sequence for AGA, AGL, ALSOA, ARSA, ARSB, ASAH1, ATG5, ATG7, CGI58, CLCN5, CLN3, CLN5, CLN6, CLN8, CTNS, CTSA, CTSK, ENO3, EPM2A, FIG4, FUCA1, G6PC, G6PT (i.e., G6PT1, G6PT2, G6PT3), GAA, GALC, GALNS, GBA, GDE, GEB1, GLA, GLB1, GM2A, GNPTAB, GNPTG, GNS, GUSB, GYG1, GYS1, GYS2, HEXA, HEXB, HGSNAT, HYAL1, IDS, IDUA, LAMP2, LIPA, MAN2B1, MANBA, MCOLN1, mTORC1, NAGA, NAGLU, NEU1, NHLRC1, NPC1, NPC1, NPC2, OCRL, PNPLA2, PPT1, PSAP, PFKM, PHKA2, PHK, PHKG2, PHKA1, PGAM2, PRKAG2, SGSH, SLC17A5, SLC37A4, SLC38A9, SLC9A6, SMPD1, SUMF1, and TPP1.

In an aspect, gene therapy can comprise administering to the subject an isolated nucleic acid molecule encoding a native or recombinant protein, such as, for example, alpha-glucosidase (GAA), α-galactosidase A (GLA), acid β-glucosidase, α-L-iduronidase, iduronate 2-sulfatase, lysosomal acid lipase, liver glycogen synthase, muscle glycogen synthase, glucose-6-phosphatase α, glucose-6-phosphate transporter, α-glucosidase, glycogen debranching enzyme, glycogen branching enzyme, muscle glycogen phosphorylase, liver glycogen phosphorylase, muscle phosphofructose kinase, phosphorylase kinase (α2 subunit), phosphorylase kinase (β subunit), phosphorylase kinase (γ subunit), phosphorylase kinase (α1 subunit), muscle phosphoglycerate mutase, glucose transporter 2, aldolase A, γ2-subunit of AMP-activated protein kinase (AMPK), β-enolase, glycogenin-1, dystrophin, factor VIII, lysosomal-associated membrane protein 2, laforin, or malin, or a fragment thereof.

In an aspect, gene therapy can comprise using a viral vector or a non-viral vector. In an aspect, a disclosed non-viral vector can be a polymer-based vector, a peptide-based vector, a lipid nanoparticle, a solid lipid nanoparticle, or a cationic lipid-based vector. In an aspect, a disclosed viral vector can be an adenovirus vector, an AAV vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

In an aspect, a disclosed viral vector can be an adeno-associated virus (AAV) vector In an aspect, a disclosed AAV vector can include naturally isolated serotypes including, but not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, an AAV capsid can be a chimera either created by capsid evolution or by rational capsid engineering from a naturally isolated AAV variants to capture desirable serotype features such as enhanced or specific tissue tropism and/or a host immune response escape. Naturally isolated AAV variants include, but not limited to, AAV-DJ, AAV-HAE1, AAV-HAE2, AAVM41, AAV-1829, AAV2 Y/F, AAV2 TN, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, a disclosed AAV vector can be AAV-Rh74 or a related variant (e.g., capsid variants like RHM4-1). In an aspect, a disclosed AAV vector can be AAV8. In an aspect, a disclosed AAV vector can be AAVhum.8. In an aspect, a disclosed AAV vector can be a self-complementary AAV.

In an aspect, a disclosed vector or a disclosed isolated nucleic acid molecule can be delivered to the subject's liver, heart, skeletal muscle, smooth muscle, CNS, PNS, or a combination thereof. In an aspect, a disclosed vector can be delivered to the subject's liver.

In an aspect, a disclosed nucleic acid molecule and/or a disclosed vector can comprise one or more disclosed regulatory sequences. Regulatory sequences are known to the art and include, but are not limited to, promoters, enhancers, and other expression control elements. In an aspect, a disclosed viral vector, such as an AAV vector, can comprise one or more promoters such as ubiquitous promoters, immunotolerant promoters, constitutive promoters, and/or tissue-specific promoters.

In an aspect, ERT can comprise administering to the subject a native or recombinant protein, such as, for example, alpha-glucosidase (GAA), α-galactosidase A (GLA), acid β-glucosidase, α-L-iduronidase, iduronate 2-sulfatase, lysosomal acid lipase, liver glycogen synthase, muscle glycogen synthase, glucose-6-phosphatase α, glucose-6-phosphate transporter, α-glucosidase, glycogen debranching enzyme, glycogen branching enzyme, muscle glycogen phosphorylase, liver glycogen phosphorylase, muscle phosphofructose kinase, phosphorylase kinase (α2 subunit), phosphorylase kinase (β subunit), phosphorylase kinase (γ subunit), phosphorylase kinase (α1 subunit), muscle phosphoglycerate mutase, glucose transporter 2, aldolase A, γ2-subunit of AMP-activated protein kinase (AMPK), β-enolase, glycogenin-1, dystrophin, factor VIII, lysosomal-associated membrane protein 2, laforin, or malin, or a fragment thereof. In an aspect, a disclosed encoded protein can comprise one or more modifications to improve expression or efficacy of that protein in a subject, such as, for example, a human patient (e.g., codon optimization). Modifications to proteins and fragments thereof are known to the art.

In an aspect, a disclosed method of detecting a Glycogen Storage Disease (GSD) biomarker can comprise administering one or more disclosed immune modulators. Immune modulators are known in the art. Immune modulators can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGF1R antibodies, an IL2 mutein, a BTK inhibitor, or a combination thereof. In an asepct, a disclosed immune modulator can comprise siltuximab. In an aspect, a disclosed immune modulator can comprise one or more Treg (regulatory T cells) infusions (e.g., antigen specific Treg cells to AAV). In an aspect, a disclosed immune modulator can be SVP-Rapamycin. In an aspect, a disclosed immune modulator can comprise bortezomib alone or in combination with methotrexate.

In an aspect, a disclosed method of detecting a Glycogen Storage Disease (GSD) biomarker can comprise administering a small molecule. In an aspect, a disclosed small molecule cannot traverse the blood-brain barrier in a meaningful quantity but is nonetheless therapeutic for a subject having a GSD. In an aspect, a disclosed small molecule can traverse the blood-brain barrier in a meaningful quantity to treat a GSD.

In an aspect, a disclosed method of detecting a Glycogen Storage Disease (GSD) biomarker can comprise SRT. In an aspect, SRT can comprise administering one or more small molecule inhibitors of biosynthesis to reduce the concentration of accumulating substrate to a level where the residual degradative enzymes can maintain homeostasis.

Disclosed herein is a method of detecting a Glycogen Storage Disease (GSD) biomarker in a subject, the method comprising obtaining a biosample from a subject suspected of having a GSD; determining the level of one or more cellular metabolites in the biosample; identifying those cellular metabolites that are differentially present in the biosample when compared to that of a reference biosample; wherein those differentially present cellular metabolites are biomarkers of a GSD.

In an aspect, determining the level of the one or more cellular metabolites in a biosample can comprise using mass spectrometry, ultra-performance liquid chromatography (UPLC), high-performance liquid chromatography (HPLC), mass spectrometry in conjunction with UPLC, LC/MS/MS, ELISA, Western blots, or any combination thereof. The skilled person can identify the method or technique that is most suitable for a given biosample. In an aspect, the same method or technique can be used for one or more biosamples or for all biosamples. In an aspect, a different method or technique can be used for one or more biosamples or for all biosamples.

In an aspect, a subject suspected of having a GSD can be an adult, a young adult, an adolescent, a child, or a newborn infant. In an aspect, a subject suspected of having a GSD can be a newborn infant. In an aspect, a subject can be a male or a female.

In an aspect, a biosample can comprise EDTA, blood, serum, or plasma.

In an aspect of a method of detecting a GSD biomarker, a disclosed reference biosample can comprise a biosample from a subject not having a GSD or can comp;rie an aggregate of biosamples from subjects not having a GSD. In an aspect, a disclosed reference biosample can comprise a biosample from the subject prior to the onset of a GSD.

In an aspect, a disclosed method of detecting a GSD biomarker can comprise obtaining a reference biosample. For example, in an aspect, obtaining a reference biosample can comprise obtaining a biosample from a subject not having a GSD and determining the level of one or more cellular metabolites in the biosample. In an aspect, obtaining a reference can comprise obtaining a biosample from subjects not having a GSD and determining the level of one or more cellular metabolites in the biosamples.

In an aspect, determining the level of one or more cellular metabolites in a reference biosample can comprise using mass spectrometry, ultra-performance liquid chromatography (UPLC), high-performance liquid chromatography (HPLC), mass spectrometry in conjunction with UPLC, LC/MS/MS, ELISA, Western blots, or any combination thereof.

In an aspect, the one or more differentially present cellular metabolites can be associated with impaired glycogenolysis, impaired gluconeogenesis, impaired ketogenesis, or any combination thereof. In an aspect, the one or more differentially present cellular metabolites can be associated with lactic acidosis, hyperuricemia, hyperlipidemia, or any combination thereof. In an aspect, the one or more differentially present cellular metabolites can be associated with impaired glycogenolysis, impaired gluconeogenesis, impaired ketogenesis, lactic acidosis, hyperuricemia, hyperlipidemia, or any combination thereof.

In an aspect, the one or more cellular metabolites can comprise 1,5-anhydroglucitol (1,5 AG), 25-hydroxyvitamin D, albumin, adiponectin, analytes associated with CLIX score, analytes associated with HOMA (Homeostasis Model Assessment) score, analytes associated with IR score, anti-islet cell cytoplasmic (anti-ICA) auto-antibodies, C-peptide, cystatin C, D-mannose, estimated glomerular filtration rate (eGFR), F2-isoprostanes, ferritin, fibrinogen, free fatty acids, fructosamine, gamma-glutamic transferase (GGT), glucose, glutamic acid decarboxylase (anti-GAD) auto-antibodies, glycation gap (glycosylation gap), HDL, HDL-c, hemoglobin (Hb) A1c, homocysteine, insulin, intact pro-insulin, LDL, LDL-c, leptin, leptin/adiponectin ratio, linoleoyl glycerophosphocholine (L-GPC), lipoprotein-associated phospholipase A2 (Lp-PLA2), mannose binding lectin (MBL) activity, mannose, mannose-binding lectin (MBL) amount, myeloperoxidase, oleic acid (OA), pro-insulin, serum amylase, serum cholinesterase, serum creatinine, triglycerides, TSH, uric acid, vitamin B12, and any combination thereof. In an aspect, one or more of the cellular metabolites can be differentially present. In an aspect, a combination of cellular metabolites can be differentially present.

In an aspect, the one or more cellular metabolites can comprise 1,5-AG, glucose, uric acid, lactate, triglycerides, and any combination thereof. In an aspect, the differentially present cellular metabolite can comprise 1,5-AG. In an aspect, the one or more cellular metabolites can comprise 1,5-AG, serum cholinesterase, albumin, and any combination thereof.

In an aspect of a disclosed method of detecting a GSD biomarker, a subject suspected of having a GSD can have a higher level of 1,5-AG than does a subject not having a GSD. In an aspect, the higher level of 1,5-AG in a subject suspected of having a GSD can be associated with a Z-score of at least +1.5, at least +2, or more than +2. In an aspect, the differential level of 1,5-AG can comprise at least a Cohen's d effect size of at least 0.2, at least 0.5, at least 0.8, or greater than 0.8. In an aspect, the differential level of 1,5-AG can comprise at least a Cohen's d effect size of at least 1, at least 2, at least 3, or greater than 3.

In an aspect of a disclosed method of detecting a GSD biomarker, one or more differentially present cellular metabolites can be associated with a Z-score of at least +1.5, at least +2, or more than +2, or one or more differentially present cellular metabolites can be associated with a Z-score of at least -1.5, at least -2, or more than -2. In an aspect, the differential level of one or more cellular metabolites can comprise at least a Cohen's d effect size of at least 0.2, at least 0.5, at least 0.8, or greater than 0.8. In an aspect, the differential level of one or more cellular metabolites can comprise at least a Cohen's d effect size of at least 1, at least 2, at least 3, or greater than 3.

In an aspect, a disclosed method can comprise diagnosing a subject suspected of having a GSD with having a GSD. GSDs are known to those skilled in the art. In an aspect, a disclosed subject can have GSD I, GSD II, GSD III, GSD IV, GSD V, GSD VI, GSD VII, GSD IX, GSD XI, GSD XII, GSD XIII, GSD IX, GSD XIV, or GSD XV. In an aspect, a disclosed subject can have GSD III, GSD VI, GSD IX, or Fanconi-Bickel syndrome. In an aspect, a disclosed subject can have GSD Ia or GSD Ib.

In an aspect, disclosed method can comprise treating the subject diagnosed with having a GSD.

In an aspect, treating the subject can comprise administering to the subject one or more agents that modulate the level of one or more differentially present cellular metabolites. In an aspect, a disclosed agent can target G6PT dysfunction of G6PT deficiency. In an aspect, treating the subject cam comprise administering to the subject one or more agents that target renal sodium glucose co-transporter 2 (SGLT2), such as, for example, empagliflozin, canagliflozin, dapagliflozin, ipragliflozin, luseogliflozin, and Tofogliflozin. In an aspect, the SGLT2 inhibitor can be empagliflozin.

In an aspect of a disclosed method, treating a subject can comprise administering to the subject to the subject enzyme replacement therapy, gene therapy, mRNA therapy, small molecule therapy, or any combination thereof. In an aspect of a disclosed method, treating a subject can comprise administering to the subject a combination of one or more agents with ERT, gene therapy, mRNA therapy, small molecule therapy, SRT, or any combination thereof.

In an aspect of a disclosed method, treating a subject can comprise implementing a change in the subject's dietary intake of carbohydrates. In an aspect, a change in a subject's dietary intake of carbohydrates can comprise adding carbohydrates to the subject's diet, or removing carbohydrates from the subject's diet, or changing the type of carbohydrates in the subject's diet, or changing the frequency of carbohydrates consumed by the subject.

In an aspect of a disclosed method, treating a subject can comprise administering cornstarch to the subject. Cornstarch is known to the art and can comprise uncooked cornstarch, modified cornstarch, a cornstarch derivative, a cornstarch alternative, or any combination thereof. In an aspect, cornstarch can comprise Glycosade . In an aspect of a disclosed method, treating a subject can comprise administering glycoside to the subject.

In an aspect, a subject's treatment can comprise ingesting one or more anaplerotic agents (e.g., medium-even-chain triglycerides (MCT), triheptanoin, etc.). In an aspect, the one or more anaplerotic agents can reduce the amount of cornstarch and/or carbohydrates required by the subject to, for example, maintain metabolic control.

In an aspect, any step in the disclosed method can be repeated one or more times. In an aspect, one or more steps in the disclosed method can be repeated one or more times.

In an aspect, a step of a disclosed method can be modified. For example, in an aspect, modifying the method can comprise modifying or changing one or more features or aspects of one or more steps of a disclosed method. For example, in an aspect, a disclosed method can be altered by changing the amount of one or more of the disclosed agents, by changing the route or frequency of administration of one or more of the disclosed agents, or by changing the duration of time one or more of the disclosed agents are administered to a subject.

In an aspect of a disclosed method, gene therapy can comprise administering to the subject an isolated nucleic acid molecule encoding a protein that is deficient and/or impaired in the subject such as, for example, a deficient and/or impaired protein having a role in glycogenolysis and/or gluconeogenesis. In an aspect, a disclosed isolated nucleic acid can comprise the sequence for AGA, AGL, ALSOA, ARSA, ARSB, ASAH1, ATG5, ATG7, CGI58, CLCN5, CLN3, CLN5, CLN6, CLN8, CTNS, CTSA, CTSK, ENO3, EPM2A, FIG4, FUCA1, G6PC, G6PT (i.e., G6PT1, G6PT2, G6PT3), GAA, GALC, GALNS, GBA, GDE, GEB1, GLA, GLB1, GM2A, GNPTAB, GNPTG, GNS, GUSB, GYG1, GYS1, GYS2, HEXA, HEXB, HGSNAT, HYAL1, IDS, IDUA, LAMP2, LIPA, MAN2B1, MANBA, MCOLN1, mTORC1, NAGA, NAGLU, NEU1, NHLRC1, NPC1, NPC1, NPC2, OCRL, PNPLA2, PPT1, PSAP, PFKM, PHKA2, PHK, PHKG2, PHKA1, PGAM2, PRKAG2, SGSH, SLC17A5, SLC37A4, SLC38A9, SLC9A6, SMPD1, SUMF1, and TPP1.

In an aspect, gene therapy can comprise administering to the subject an isolated nucleic acid molecule encoding a native or recombinant protein, such as, for example, alpha-glucosidase (GAA), α-galactosidase A (GLA), acid β-glucosidase, α-L-iduronidase, iduronate 2-sulfatase, lysosomal acid lipase, liver glycogen synthase, muscle glycogen synthase, glucose-6-phosphatase α, glucose-6-phosphate transporter, α-glucosidase, glycogen debranching enzyme, glycogen branching enzyme, muscle glycogen phosphorylase, liver glycogen phosphorylase, muscle phosphofructose kinase, phosphorylase kinase (α2 subunit), phosphorylase kinase (β subunit), phosphorylase kinase (γ subunit), phosphorylase kinase (α1 subunit), muscle phosphoglycerate mutase, glucose transporter 2, aldolase A, γ2-subunit of AMP-activated protein kinase (AMPK), β-enolase, glycogenin-1, dystrophin, factor VIII, lysosomal-associated membrane protein 2, laforin, or malin, or a fragment thereof.

In an aspect, gene therapy can comprise using a disclosed viral vector or a disclosed non-viral vector (discussed supra). In an aspect, a disclosed viral vector can be a disclosed adeno-associated virus (AAV) vector. Naturally isolated serotypes as well as recombinant serotypes are known to the art and discussed supra. In an aspect, a disclosed vector or a disclosed isolated nucleic acid molecule can be delivered to the subject's liver, heart, skeletal muscle, smooth muscle, CNS, PNS, or a combination thereof. In an aspect, a disclosed vector can be delivered to the subject's liver. In an aspect, a disclosed nucleic acid molecule and/or a disclosed vector can comprise one or more disclosed regulatory sequences.

In an aspect, ERT can comprise administering to the subject a native or recombinant protein, such as, for example, alpha-glucosidase (GAA), α-galactosidase A (GLA), acid β-glucosidase, α-L-iduronidase, iduronate 2-sulfatase, lysosomal acid lipase, liver glycogen synthase, muscle glycogen synthase, glucose-6-phosphatase α, glucose-6-phosphate transporter, α-glucosidase, glycogen debranching enzyme, glycogen branching enzyme, muscle glycogen phosphorylase, liver glycogen phosphorylase, muscle phosphofructose kinase, phosphorylase kinase (α2 subunit), phosphorylase kinase (β subunit), phosphorylase kinase (γ subunit), phosphorylase kinase (α1 subunit), muscle phosphoglycerate mutase, glucose transporter 2, aldolase A, γ2-subunit of AMP-activated protein kinase (AMPK), β-enolase, glycogenin-1, dystrophin, factor VIII, lysosomal-associated membrane protein 2, laforin, or malin, or a fragment thereof. In an aspect, a disclosed encoded protein can comprise one or more modifications to improve expression or efficacy of that protein in a subject, such as, for example, a human patient (e.g., codon optimization). Modifications to proteins and fragments thereof are known to the art.

In an aspect, a disclosed method of detecting a Glycogen Storage Disease (GSD) biomarker can comprise administering one or more disclosed immune modulators. Immune modulators are known in the art and are discussed supra. In an aspect, a disclosed immune modulator can comprise bortezomib, intravenous gamma globulin (IVIG), methotrexate, or any combination thereof.

In an aspect, a disclosed method of detecting a Glycogen Storage Disease (GSD) biomarker can comprise administering a disclosed small molecule. In an aspect, a disclosed small molecule cannot traverse the blood-brain barrier in a meaningful quantity but is nonetheless therapeutic for a subject having a GSD. In an aspect, a disclosed small molecule can traverse the blood-brain barrier in a meaningful quantity to treat a GSD.

In an aspect, a disclosed method of detecting a Glycogen Storage Disease (GSD) biomarker can comprise SRT. In an aspect, SRT can comprise administering one or more small molecule inhibitors of biosynthesis to reduce the concentration of accumulating substrate to a level where the residual degradative enzymes can maintain homeostasis.

### D. Methods of Diagnosing a Glycogen Storage Disease (GSD)

Disclosed herein is a method of diagnosing a subject with a Glycogen Storage Disease (GSD), the method comprising obtaining a biosample from a subject suspected of having a GSD; determining the level of 1,5-anhydroglucitol (1,5-AG) in the biosample; and when the level of 1,5-AG in the biosample of the subject suspected of having a GSD is significantly higher than that of a reference biosample, then diagnosing the subject with a GSD.

Disclosed herein a method of diagnosing a subject with a Glycogen Storage Disease (GSD), the method comprising obtaining a biosample from a subject suspected of having GSD I; determining the level of 1,5-anhydroglucitol (1,5-AG) in the biosample; and when the level of 1,5-AG in the biosample of the subject suspected of having GSD I is significantly higher than that of a reference biosample, then diagnosing the subject with a GSD Ia or Ib.

In an aspect, a subject suspected of having a GSD can have a higher level of 1,5-AG than does a subject not having a GSD. In an aspect, the higher level of 1,5-AG in a subject suspected of having a GSD can be associated with a Z-score of at least +1.5, at least +2, or more than +2. In an aspect, the differential level of 1,5-AG can comprise at least a Cohen's d effect size of at least 0.2, at least 0.5, at least 0.8, or greater than 0.8. In an aspect, the differential level of 1,5-AG can comprise at least a Cohen's d effect size of at least 1, at least 2, at least 3, or greater than 3.

In an aspect, a disclosed subject can be diagnosed as having GSD I, GSD II, GSD III, GSD IV, GSD V, GSD VI, GSD VII, GSD IX, GSD XI, GSD XII, GSD XIII, GSD IX, GSD XIV, or GSD XV. In an aspect, a disclosed subject can have GSD III, GSD VI, GSD IX, or Fanconi-Bickel syndrome. In an aspect, a disclosed subject can have GSD Ia or GSD Ib.

In an aspect, a subject suspected of having a GSD can be an adult, a young adult, an adolescent, a child, or a newborn infant. In an aspect, a subject suspected of having a GSD can be a newborn infant. In an aspect, a subject can be a male or a female.

In an aspect, determining the level of level of 1,5-AG in a biosample can comprise using mass spectrometry, ultra-performance liquid chromatography (UPLC), high-performance liquid chromatography (HPLC), mass spectrometry in conjunction with UPLC, LC/MS/MS, ELISA, Western blots, or any combination thereof. The skilled person can identify the method or technique that is most suitable for a given biosample. In an aspect, the same method or technique can be used for one or more biosamples or for all biosamples. In an aspect, a different method or technique can be used for one or more biosamples or for all biosamples.

In an aspect of a method of detecting a GSD biomarker, a disclosed reference biosample can comprise a biosample from a subject not having a GSD or can comprise an aggregate of biosamples from subjects not having a GSD. In an aspect, a disclosed reference biosample can comprise a biosample from the subject prior to the onset of a GSD.

In an aspect, a disclosed method of detecting a GSD biomarker can comprise obtaining a reference biosample. For example, in an aspect, obtaining a reference biosample can comprise obtaining a biosample from a subject not having a GSD and determining the level of 1,5-AG in the biosample. In an aspect, obtaining a reference can comprise obtaining a biosample from subjects not having a GSD and determining the level of 1,5-AG in the biosamples.

In an aspect, a disclosed reference biosample can comprise a biosample from a subject that is an adult, a young adult, an adolescent, a child, or a newborn infant. In an aspect, a disclosed reference biosample can comprise biosamples from subjects that are adults, young adults, adolescents, children, or newborn infants.

In an aspect, determining the level of level of 1,5-AG in a reference biosample can comprise using mass spectrometry, ultra-performance liquid chromatography (UPLC), high-performance liquid chromatography (HPLC), mass spectrometry in conjunction with UPLC, LC/MS/MS, ELISA, Western blots, or any combination thereof.

In an aspect, a disclosed biosample can comprise EDTA, blood, serum, or plasma.

In an aspect, a disclosed method can comprise determining the level of one or more cellular metabolites and comparing the level of the one or more cellular metabolites to that of a reference biosample to identify one or more differentially present cellular metabolites.

In an aspect, the one or more differentially present cellular metabolites can be associated with impaired glycogenolysis, impaired gluconeogenesis, impaired ketogenesis, or any combination thereof. In an aspect, the one or more differentially present cellular metabolites can be associated with lactic acidosis, hyperuricemia, hyperlipidemia, or any combination thereof. In an aspect, the one or more differentially present cellular metabolites can be associated with impaired glycogenolysis, impaired gluconeogenesis, impaired ketogenesis, lactic acidosis, hyperuricemia, hyperlipidemia, or any combination thereof.

In an aspect, the one or more cellular metabolites can comprise 25-hydroxyvitamin D, albumin, adiponectin, analytes associated with CLIX score, analytes associated with HOMA (Homeostasis Model Assessment) score, analytes associated with IR score, anti-islet cell cytoplasmic (anti-ICA) auto-antibodies, C-peptide, cystatin C, D-mannose, estimated glomerular filtration rate (eGFR), F2-isoprostanes, ferritin, fibrinogen, free fatty acids, fructosamine, gamma-glutamic transferase (GGT), glucose, glutamic acid decarboxylase (anti-GAD) auto-antibodies, glycation gap (glycosylation gap), HDL, HDL-c, hemoglobin (Hb) A1c, homocysteine, insulin, intact pro-insulin, LDL, LDL-c, leptin, leptin/adiponectin ratio, linoleoyl glycerophosphocholine (L-GPC), lipoprotein-associated phospholipase A2 (Lp-PLA2), mannose binding lectin (MBL) activity, mannose, mannose-binding lectin (MBL) amount, myeloperoxidase, oleic acid (OA), pro-insulin, serum amylase, serum cholinesterase, serum creatinine, triglycerides, TSH, uric acid, vitamin B12, and any combination thereof. In an aspect, one or more of the cellular metabolites can be differentially present. In an aspect, a combination of cellular metabolites can be differentially present.

In an aspect, the one or more cellular metabolites can comprise glucose, uric acid, lactate, triglycerides, and any combination thereof. In an aspect, the one or more cellular metabolites can comprise serum cholinesterase, albumin, and any combination thereof.

In an aspect, one or more differentially present cellular metabolites can be associated with a Z-score of at least +1.5, at least +2, or more than +2, or one or more differentially present cellular metabolites can be associated with a Z-score of at least −1.5, at least −2, or more than −2. In an aspect, the differential level of one or more cellular metabolites can comprise at least a Cohen's d effect size of at least 0.2, at least 0.5, at least 0.8, or greater than 0.8. In an aspect, the differential level of one or more cellular metabolites can comprise at least a Cohen's d effect size of at least 1, at least 2, at least 3, or greater than 3.

In an aspect, disclosed method can comprise treating the subject diagnosed with having a GSD.

In an aspect, treating the subject can comprise administering to the subject one or more agents that modulate the level of one or more differentially present cellular metabolites. In an aspect, a disclosed agent can target G6PT dysfunction of G6PT deficiency. In an aspect, treating the subject cam comprise administering to the subject one or more agents that target renal sodium glucose co-transporter 2 (SGLT2), such as, for example, empagliflozin, canagliflozin, dapagliflozin, ipragliflozin, luseogliflozin, and Tofogliflozin. In an aspect, the SGLT2 inhibitor can be empagliflozin.

In an aspect of a disclosed method, treating a subject can comprise administering to the subject to the subject ERT, gene therapy, mRNA therapy, small molecule therapy, SRT, or any combination thereof. In an aspect of a disclosed method, treating a subject can comprise administering to the subject a combination of one or more agents with ERT, gene therapy, mRNA therapy, small molecule therapy, SRT, or any combination thereof.

In an aspect of a disclosed method, treating a subject can comprise implementing a change in the subject's dietary intake of carbohydrates. In an aspect, a change in a subject's dietary intake of carbohydrates can comprise adding carbohydrates to the subject's diet, or removing carbohydrates from the subject's diet, or changing the type of carbohydrates in the subject's diet, or changing the frequency of carbohydrates consumed by the subject.

In an aspect, a disclosed method can comprise administering cornstarch to the subject. Cornstarch is known to the art and can comprise uncooked cornstarch, modified cornstarch, a cornstarch derivative, a cornstarch alternative, or any combination thereof. In an aspect, cornstarch can comprise Glycosade®. In an aspect, a disclosed method can comprise administering glycoside to the subject.

In an aspect, a subject's treatment can comprise ingesting one or more anaplerotic agents (e.g., medium-even-chain triglycerides (MCT), triheptanoin, etc.). In an aspect, the one or more anaplerotic agents can reduce the amount of cornstarch and/or carbohydrates required by the subject to, for example, maintain metabolic control.

In an aspect, any step in the disclosed method can be repeated one or more times. In an aspect, one or more steps in the disclosed method can be repeated one or more times.

In an aspect, a step of a disclosed method can be modified. For example, in an aspect, modifying the method can comprise modifying or changing one or more features or aspects of one or more steps of a disclosed method. For example, in an aspect, a disclosed method can be altered by changing the amount of one or more of the disclosed agents, by changing the route or frequency of administration of one or more of the disclosed agents, or by changing the duration of time one or more of the disclosed agents are administered to a subject.

In an aspect of a disclosed method, gene therapy can comprise administering to the subject an isolated nucleic acid molecule encoding a protein that is deficient and/or impaired in the subject such as, for example, a deficient and/or impaired protein having a role in glycogenolysis and/or gluconeogenesis. In an aspect, a disclosed isolated nucleic acid can comprise the sequence for AGA, AGL, ALSOA, ARSA, ARSB, ASAH1, ATG5, ATG7, CGI58, CLCN5, CLN3, CLN5, CLN6, CLN8, CTNS, CTSA, CTSK, ENO3, EPM2A, FIG4, FUCA1, G6PC, G6PT (i.e., G6PT1, G6PT2, G6PT3), GAA, GALC, GALNS, GBA, GDE, GEB1, GLA, GLB1, GM2A, GNPTAB, GNPTG, GNS, GUSB, GYG1, GYS1, GYS2, HEXA, HEXB, HGSNAT, HYAL1, IDS, IDUA, LAMP2, LIPA, MAN2B1, MANBA, MCOLN1, mTORC1, NAGA, NAGLU, NEU1, NHLRC1, NPC1, NPC1, NPC2, OCRL, PNPLA2, PPT1, PSAP, PFKM, PHKA2, PHK, PHKG2, PHKA1, PGAM2, PRKAG2, SGSH, SLC17A5, SLC37A4, SLC38A9, SLC9A6, SMPD1, SUMF1, and TPP1.

In an aspect, gene therapy can comprise administering to the subject an isolated nucleic acid molecule encoding a native or recombinant protein, such as, for example, alpha-glucosidase (GAA), α-galactosidase A (GLA), acid β-glucosidase, α-L-iduronidase, iduronate 2-sulfatase, lysosomal acid lipase, liver glycogen synthase, muscle glycogen synthase, glucose-6-phosphatase α, glucose-6-phosphate transporter, α-glucosidase, glycogen debranching enzyme, glycogen branching enzyme, muscle glycogen phosphorylase, liver glycogen phosphorylase, muscle phosphofructose kinase, phosphorylase kinase (α2 subunit), phosphorylase kinase (β subunit), phosphorylase kinase (γ subunit), phosphorylase kinase (α1 subunit), muscle phosphoglycerate mutase, glucose transporter 2, aldolase A, γ2-subunit of AMP-activated protein kinase (AMPK), β-enolase, glycogenin-1, dystrophin, factor VIII, lysosomal-associated membrane protein 2, laforin, or malin, or a fragment thereof.

In an aspect, gene therapy can comprise using a disclosed viral vector or a disclosed non-viral vector (discussed supra). In an aspect, a disclosed viral vector can be a disclosed adeno-associated virus (AAV) vector. Naturally isolated serotypes as well as recombinant serotypes are known to the art and discussed supra. In an aspect, a disclosed vector or a disclosed isolated nucleic acid molecule can be delivered to the subject's liver, heart, skeletal muscle, smooth muscle, CNS, PNS, or a combination thereof. In an aspect, a disclosed vector can be delivered to the subject's liver. In an aspect, a disclosed nucleic acid molecule and/or a disclosed vector can comprise one or more disclosed regulatory sequences.

In an aspect, enzyme replacement therapy can comprise administering to the subject a native or recombinant protein, such as, for example, alpha-glucosidase (GAA), α-galactosidase A (GLA), acid β-glucosidase, α-L-iduronidase, iduronate 2-sulfatase, lysosomal acid lipase, liver glycogen synthase, muscle glycogen synthase, glucose-6-phosphatase α, glucose-6-phosphate transporter, α-glucosidase, glycogen debranching enzyme, glycogen branching enzyme, muscle glycogen phosphorylase, liver glycogen phosphorylase, muscle phosphofructose kinase, phosphorylase kinase (α2 subunit), phosphorylase kinase (β subunit), phosphorylase kinase (γ subunit), phosphorylase kinase (α1 subunit), muscle phosphoglycerate mutase, glucose transporter 2, aldolase A, γ2-subunit of AMP-activated protein kinase (AMPK), β-enolase, glycogenin-1, dystrophin, factor VIII, lysosomal-associated membrane protein 2, laforin, or malin, or a fragment thereof. In an aspect, a disclosed encoded protein can comprise one or more modifications to improve expression or efficacy of that protein in a subject, such as, for example, a human patient (e.g., codon optimization). Modifications to proteins and fragments thereof are known to the art.

In an aspect, a disclosed method of detecting a Glycogen Storage Disease (GSD) biomarker can comprise administering one or more disclosed immune modulators. Immune modulators are known in the art and discussed supra. In an aspect, a disclosed immune modulator can comprise bortezomib, intravenous gamma globulin (IVIG), methotrexate, or any combination thereof. In an aspect, a disclosed immune modulator can comprise bortezomib alone or in combination with methotrexate.

In an aspect, a disclosed method of diagnosing a Glycogen Storage Disease (GSD) can comprise administering a small molecule. In an aspect, a disclosed small molecule cannot traverse the blood-brain barrier in a meaningful quantity but is nonetheless therapeutic for a subject having a GSD. In an aspect, a disclosed small molecule can traverse the blood-brain barrier in a meaningful quantity to treat a GSD.

In an aspect, a disclosed method of diagnosing a Glycogen Storage Disease (GSD) can comprise SRT. In an aspect, SRT can comprise administering one or more small molecule inhibitors of biosynthesis to reduce the concentration of accumulating substrate to a level where the residual degradative enzymes can maintain homeostasis.

### E. Methods of Treating a Glycogen Storage Disease (GSD)

Disclosed herein is a method of treating a subject having a Glycogen Storage Disease (GSD), the method comprising obtaining a post-treatment biosample from a subject having a GSD; determining the level of 1,5-anhydroglucitol (1,5-AG) in the biosample; and if the post-treatment level of 1,5-AG represents an improvement over a pre-treatment level of 1,5-AG, or if the post-treatment level of 1,5-AG is within an acceptable range of a reference level, then continuing to administer the treatment.

Disclosed herein is a method of treating a subject having a Glycogen Storage Disease (GSD), the method comprising obtaining a post-treatment biosample from a subject having a GSD; determining the level of 1,5-anhydroglucitol (1,5-AG) in the biosample; and if the post-treatment level of 1,5-AG represents an improvement over a pre-treatment level of 1,5-AG, or if the post-treatment level of 1,5-AG is within an acceptable range of a reference level, then confirming that the treatment is effective.

In an aspect, a disclosed treatment can be considered ineffective when the post-treatment level of 1,5-AG is higher than the pre-treatment level of 1,5-AG, or when the post-treatment level of 1,5-AG is not within an acceptable range of the reference level of 1,5-AG.

In an aspect, an effective treatment can be repeated one or more times. In an aspect, an ineffective treatment can be modified in one or more ways.

In an aspect, a subject having a GSD can be an adult, a young adult, an adolescent, a child, or a newborn infant. In an aspect, a subject having a GSD can be a newborn infant. In an aspect, a subject can be a male or a female.

In an aspect, the GSD can be GSD I, GSD II, GSD III, GSD IV, GSD V, GSD VI, GSD VII, GSD IX, GSD XI, GSD XII, GSD XIII, GSD IX, GSD XIV, or GSD XV. In an aspect, the GSD can be GSD III, GSD VI, GSD IX, or Fanconi-Bickel syndrome. In an aspect, the GSD can be GSD Ia or GSD Ib.

In an aspect, determining the level of level of 1,5-AG in a biosample can comprise using mass spectrometry, ultra-performance liquid chromatography (UPLC), high-performance liquid chromatography (HPLC), mass spectrometry in conjunction with UPLC, LC/MS/MS, ELISA, Western blots, or any combination thereof. The skilled person can identify the method or technique that is most suitable for a given biosample. In an aspect, the same method or technique can be used for one or more biosamples or for all biosamples. In an aspect, a different method or technique can be used for one or more biosamples or for all biosamples.

In an aspect of a method of detecting a GSD biomarker, a disclosed reference biosample can comprise a biosample from a subject not having a GSD or can comprise an aggregate of biosamples from subjects not having a GSD. In an aspect, a disclosed reference biosample can comprise a biosample from a subject that is an adult, a young adult, an adolescent, a child, or a newborn infant. In an aspect, a disclosed reference biosample can comprise biosamples from subjects that are adults, young adults, adolescents, children, or newborn infants.

In an aspect, a disclosed method of detecting a GSD biomarker can comprise obtaining a reference biosample. For example, in an aspect, obtaining a reference biosample can comprise obtaining a biosample from a subject not having a GSD and determining the level of 1,5-AG in the biosample. In an aspect, obtaining a reference can comprise obtaining a biosample from subjects not having a GSD and determining the level of 1,5-AG in the biosamples.

In an aspect, determining the level of level of 1,5-AG in a reference biosample can comprise using mass spectrometry, ultra-performance liquid chromatography (UPLC), high-performance liquid chromatography (HPLC), mass spectrometry in conjunction with UPLC, LC/MS/MS, ELISA, Western blots, or any combination thereof.

In an aspect, a disclosed biosample can comprise EDTA, blood, serum, or plasma.

In an aspect, a disclosed method can comprise obtaining a biosample from the subject prior to treatment and detecting the level of 1,5-AG in the pre-treatment biosample.

In an aspect of a disclosed method, the post-treatment level of 1,5-AG can represent an improvement over a pre-treatment level when the post-treatment level is lower than the pre-treatment level. In an aspect, the post-treatment level of 1,5-AG can represent an improvement over a pre-treatment level when the post-treatment level is more similar to a reference level than to the pre-treatment expression level.

A disclosed method can comprise determining the level of one or more cellular metabolites in the post-treatment biosample and comparing the level of the one or more cellular metabolites to that of the pre-treatment biosample or to that of the reference biosample to identify one or more differentially present cellular metabolites.

In an aspect, determining the level of the one or more cellular metabolites a biosample can comprise using mass spectrometry, ultra-performance liquid chromatography (UPLC), high-performance liquid chromatography (HPLC), mass spectrometry in conjunction with UPLC, LC/MS/MS, ELISA, Western blots, or any combination thereof. The skilled person can identify the method or technique that is most suitable for a given biosample. In an aspect, the same method or technique can be used for one or more biosamples or for all biosamples. In an aspect, a different method or technique can be used for one or more biosamples or for all biosamples.

In an aspect, the one or more differentially present cellular metabolites can be associated with impaired glycogenolysis, impaired gluconeogenesis, impaired ketogenesis, or any combination thereof. In an aspect, the one or more differentially present cellular metabolites can be associated with lactic acidosis, hyperuricemia, hyperlipidemia, or any combination thereof. In an aspect, the one or more differentially present cellular metabolites can be associated with impaired glycogenolysis, impaired gluconeogenesis, impaired ketogenesis, lactic acidosis, hyperuricemia, hyperlipidemia, or any combination thereof.

In an aspect, the one or more cellular metabolites can comprise 25-hydroxyvitamin D, albumin, adiponectin, analytes associated with CLIX score, analytes associated with HOMA (Homeostasis Model Assessment) score, analytes associated with IR score, anti-islet cell cytoplasmic (anti-ICA) auto-antibodies, C-peptide, cystatin C, D-mannose, estimated glomerular filtration rate (eGFR), F2-isoprostanes, ferritin, fibrinogen, free fatty acids, fructosamine, gamma-glutamic transferase (GGT), glucose, glutamic acid decarboxylase (anti-GAD) auto-antibodies, glycation gap (glycosylation gap), HDL, HDL-c, hemoglobin (Hb) A1c, homocysteine, insulin, intact pro-insulin, LDL, LDL-c, leptin, leptin/adiponectin ratio, linoleoyl glycerophosphocholine (L-GPC), lipoprotein-associated phospholipase A2 (Lp-PLA2), mannose binding lectin (MBL) activity, mannose, mannose-binding lectin (MBL) amount, myeloperoxidase, oleic acid (OA), pro-insulin, serum amylase, serum cholinesterase, serum creatinine, triglycerides, TSH, uric acid, vitamin B12, and any combination thereof. In an aspect, one or more of the cellular metabolites can be differentially present. In an aspect, a combination of cellular metabolites can be differentially present.

In an aspect, the one or more cellular metabolites can comprise glucose, uric acid, lactate, triglycerides, and any combination thereof. In an aspect, the one or more cellular metabolites can comprise serum cholinesterase, albumin, and any combination thereof.

In an aspect, the one or more cellular metabolites can comprise 1,5-AG, glucose, uric acid, lactate, triglycerides, and any combination thereof. In an aspect, the differentially present cellular metabolite can comprise 1,5-AG. In an aspect, the one or more cellular metabolites can comprise 1,5-AG, serum cholinesterase, albumin, and any combination thereof.

In an aspect, one or more differentially present cellular metabolites can be associated with a Z-score of at least +1.5, at least +2, or more than +2, or one or more differentially present cellular metabolites can be associated with a Z-score of at least −1.5, at least −2, or more than −2. In an aspect, the differential level of one or more cellular metabolites can comprise at least a Cohen's d effect size of at least 0.2, at least 0.5, at least 0.8, or greater than 0.8. In an aspect, the differential level of one or more cellular metabolites can comprise at least a Cohen's d effect size of at least 1, at least 2, at least 3, or greater than 3.

In an aspect, disclosed method can comprise treating the subject diagnosed with having a GSD.

In an aspect, treating the subject can comprise administering to the subject one or more agents that modulate the level of one or more differentially present cellular metabolites. In an aspect, a disclosed agent can target G6PT dysfunction of G6PT deficiency. In an aspect, treating the subject cam comprise administering to the subject one or more agents that target renal sodium glucose co-transporter 2 (SGLT2), such as, for example, empagliflozin, canagliflozin, dapagliflozin, ipragliflozin, luseogliflozin, and Tofogliflozin. In an aspect, the SGLT2 inhibitor can be empagliflozin.

In an aspect of a disclosed method, treating a subject can comprise administering to the subject to the subject ERT, gene therapy, mRNA therapy, small molecule therapy, SRT, or any combination thereof. In an aspect of a disclosed method, treating a subject can comprise administering to the subject a combination of one or more agents with ERT, gene therapy, mRNA therapy, small molecule therapy, SRT, or any combination thereof.

In an aspect of a disclosed method, treating a subject can comprise implementing a change in the subject's dietary intake of carbohydrates. In an aspect, a change in a subject's dietary intake of carbohydrates can comprise adding carbohydrates to the subject's diet, or removing carbohydrates from the subject's diet, or changing the type of carbohydrates in the subject's diet, or changing the frequency of carbohydrates consumed by the subject.

In an aspect of a disclosed method, treating a subject can comprise administering cornstarch to the subject. Cornstarch is known to the art and can comprise uncooked cornstarch, modified cornstarch, a cornstarch derivative, a cornstarch alternative, or any combination thereof. In an aspect, cornstarch can comprise Glycosade . In an aspect of a disclosed method, treating a subject can comprise administering glycoside to the subject.

In an aspect, a subject's treatment can comprise ingesting one or more anaplerotic agents (e.g., medium-even-chain triglycerides (MCT), triheptanoin, etc.). In an aspect, the one or more anaplerotic agents can reduce the amount of cornstarch and/or carbohydrates required by the subject to, for example, maintain metabolic control.

In an aspect, any step in the disclosed method can be repeated one or more times. In an aspect, one or more steps in the disclosed method can be repeated one or more times.

In an aspect, a step of a disclosed method can be modified. For example, in an aspect, modifying the method can comprise modifying or changing one or more features or aspects of one or more steps of a disclosed method. For example, in an aspect, a disclosed method can be altered by changing the amount of one or more of the disclosed agents, by changing the route or frequency of administration of one or more of the disclosed agents, or by changing the duration of time one or more of the disclosed agents are administered to a subject.

In an aspect of a disclosed method, gene therapy can comprise administering to the subject an isolated nucleic acid molecule encoding a protein that is deficient and/or impaired in the subject such as, for example, a deficient and/or impaired protein having a role in glycogenolysis and/or gluconeogenesis. In an aspect, a disclosed isolated nucleic acid can comprise the sequence for AGA, AGL, ALSOA, ARSA, ARSB, ASAH1, ATG5, ATG7, CGI58, CLCN5, CLN3, CLN5, CLN6, CLN8, CTNS, CTSA, CTSK, ENO3, EPM2A, FIG4, FUCA1, G6PC, G6PT (i.e., G6PT1, G6PT2, G6PT3), GAA, GALC, GALNS, GBA, GDE, GEB1, GLA, GLB1, GM2A, GNPTAB, GNPTG, GNS, GUSB, GYG1, GYS1, GYS2, HEXA, HEXB, HGSNAT, HYAL1, IDS, IDUA, LAMP2, LIPA, MAN2B1, MANBA, MCOLN1, mTORC1, NAGA, NAGLU, NEU1, NHLRC1, NPC1, NPC1, NPC2, OCRL, PNPLA2, PPT1, PSAP, PFKM, PHKA2, PHK, PHKG2, PHKA1, PGAM2, PRKAG2, SGSH, SLC17A5, SLC37A4, SLC38A9, SLC9A6, SMPD1, SUMF1, and TPP1.

In an aspect, gene therapy can comprise administering to the subject an isolated nucleic acid molecule encoding a native or recombinant protein, such as, for example, alpha-glucosidase (GAA), α-galactosidase A (GLA), acid β-glucosidase, α-L-iduronidase, iduronate 2-sulfatase, lysosomal acid lipase, liver glycogen synthase, muscle glycogen synthase, glucose-6-phosphatase α, glucose-6-phosphate transporter, α-glucosidase, glycogen debranching enzyme, glycogen branching enzyme, muscle glycogen phosphorylase, liver glycogen phosphorylase, muscle phosphofructose kinase, phosphorylase kinase (α2 subunit), phosphorylase kinase (β subunit), phosphorylase kinase (γ subunit), phosphorylase kinase (α1 subunit), muscle phosphoglycerate mutase, glucose transporter 2, aldolase A, γ2-subunit of AMP-activated protein kinase (AMPK), β-enolase, glycogenin-1, dystrophin, factor VIII, lysosomal-associated membrane protein 2, laforin, or malin, or a fragment thereof.

In an aspect, gene therapy can comprise using a disclosed viral vector or a disclosed non-viral vector (discussed supra). In an aspect, a disclosed viral vector can be a disclosed adeno-associated virus (AAV) vector. Naturally isolated serotypes as well as recombinant serotypes are known to the art and discussed supra. In an aspect, a disclosed vector or a disclosed isolated nucleic acid molecule can be delivered to the subject's liver, heart, skeletal muscle, smooth muscle, CNS, PNS, or a combination thereof. In an aspect, a disclosed vector can be delivered to the subject's liver. In an aspect, a disclosed nucleic acid molecule and/or a disclosed vector can comprise one or more disclosed regulatory sequences.

In an aspect, enzyme replacement therapy can comprise administering to the subject a native or recombinant protein, such as, for example, alpha-glucosidase (GAA), α-galactosidase A (GLA), acid β-glucosidase, α-L-iduronidase, iduronate 2-sulfatase, lysosomal acid lipase, liver glycogen synthase, muscle glycogen synthase, glucose-6-phosphatase α, glucose-6-phosphate transporter, α-glucosidase, glycogen debranching enzyme, glycogen branching enzyme, muscle glycogen phosphorylase, liver glycogen phosphorylase, muscle phosphofructose kinase, phosphorylase kinase (α2 subunit), phosphorylase kinase (β subunit), phosphorylase kinase (γ subunit), phosphorylase kinase (α1 subunit), muscle phosphoglycerate mutase, glucose transporter 2, aldolase A, γ2-subunit of AMP-activated protein kinase (AMPK), β-enolase, glycogenin-1, dystrophin, factor VIII, lysosomal-associated membrane protein 2, laforin, or malin, or a fragment thereof. In an aspect, a disclosed encoded protein can comprise one or more modifications to improve expression or efficacy of that protein in a subject, such as, for example, a human patient (e.g., codon optimization). Modifications to proteins and fragments thereof are known to the art.

In an aspect, a disclosed method of treating a Glycogen Storage Disease (GSD) biomarker can comprise administering one or more disclosed immune modulators. Immune modulators are known in the art and discussed supra. In an aspect, a disclosed immune modulator can comprise bortezomib, intravenous gamma globulin (IVIG), methotrexate, or any combination thereof. In an aspect, a disclosed immune modulator can comprise bortezomib alone or in combination with methotrexate.

In an aspect, a disclosed method of treating a Glycogen Storage Disease (GSD) biomarker can comprise administering a small molecule. In an aspect, a disclosed small molecule cannot traverse the blood-brain barrier in a meaningful quantity but is nonetheless therapeutic for a subject having a GSD. In an aspect, a disclosed small molecule can traverse the blood-brain barrier in a meaningful quantity to treat a GSD.

In an aspect, a disclosed method of treating a Glycogen Storage Disease (GSD) biomarker can comprise SRT. In an aspect, SRT can comprise administering one or more small molecule inhibitors of biosynthesis to reduce the concentration of accumulating substrate to a level where the residual degradative enzymes can maintain homeostasis.

### F. Methods of Determining the Efficacy of Treatment

Disclosed herein is a method of determining the efficacy of a treatment in a subject having a Glycogen Storage Disease (GSD), the method comprising obtaining a pre-treatment biosample from a subject having a GSD and determining the level of 1,5-anhydroglucitol (1,5-AG) in the biosample; administering a treatment; obtaining a post-treatment biosample from the subject and determining the level of 1,5-anhydroglucitol (1,5-AG) in the biosample; and comparing the level of 1,5-AG in the pre-treatment biosample to the level of 1,5-AG in the post-treatment biosample to determine the subject's postprandial glucose excursion (PPGE).

In an aspect, a disclosed treatment can comprise administering cornstarch to the subject. Cornstarch is known to the art and can comprise uncooked cornstarch, modified cornstarch, a cornstarch derivative, a cornstarch alternative, or any combination thereof. In an aspect, cornstarch can comprise Glycosade®. In an aspect, a disclosed method can comprise administering glycoside to the subject.

In an aspect, a disclosed method can comprise modifying the treatment when the PPGE exceeds 70 mg/dL or when the PPGE exceeds 150 mg/dL. In an aspect, a disclosed method can comprise modifying the treatment when the PPGE is in the range 70 mg/dL to 150 mg/dL. In an aspect, modifying the treatment can comprise administering more cornstarch (e.g., uncooked cornstarch, modified cornstarch, a cornstarch derivative, a cornstarch alternative, or a combination thereof) to the subject. In an aspect, modifying the treatment can comprise administering more Glycosade® to the subject. In an aspect, modifying the treatment can comprise administering more glycoside to the subject.

In an aspect, a disclosed method can comprise determining the level of one or more cellular metabolites in the biosamples and comparing the level of the one or more cellular metabolites in the biosamples to identify one or more differentially present cellular metabolites.

In an aspect, determining the level of level of 1,5-AG in a biosample can comprise using mass spectrometry, ultra-performance liquid chromatography (UPLC), high-performance liquid chromatography (HPLC), mass spectrometry in conjunction with UPLC, LC/MS/MS, ELISA, Western blots, or any combination thereof. In an aspect, determining the level of the one or more cellular metabolites in a biosample can comprise using mass spectrometry, ultra-performance liquid chromatography (UPLC), high-performance liquid chromatography (HPLC), mass spectrometry in conjunction with UPLC, LC/MS/MS, ELISA, Western blots, or any combination thereof. The skilled person can identify the method or technique that is most suitable for a given biosample. In an aspect, the same method or technique can be used for one or more biosamples or for all biosamples. In an aspect, a different method or technique can be used for one or more biosamples or for all biosamples.

In an aspect, the disclosed one or more differentially present cellular metabolites can be associated with a Z-score of at least +1.5, at least +2, or more than +2, or one or more differentially present cellular metabolites can be associated with a Z-score of at least −1.5, at least −2, or more than −2. In an aspect, the differential level of the disclosed one or more cellular metabolites can comprise at least a Cohen's d effect size of at least 0.2, at least 0.5, at least 0.8, or greater than 0.8. In an aspect, the differential level of one or more cellular metabolites can comprise at least a Cohen's d effect size of at least 1, at least 2, at least 3, or greater than 3.

In an aspect, the one or more differentially present cellular metabolites can be associated with impaired glycogenolysis, impaired gluconeogenesis, impaired ketogenesis, or any combination thereof. In an aspect, the one or more differentially present cellular metabolites can be associated with lactic acidosis, hyperuricemia, hyperlipidemia, or any combination thereof. In an aspect, the one or more differentially present cellular metabolites can be associated with impaired glycogenolysis, impaired gluconeogenesis, impaired ketogenesis, lactic acidosis, hyperuricemia, hyperlipidemia, or any combination thereof.

In an aspect, the one or more cellular metabolites can comprise 25-hydroxyvitamin D, albumin, adiponectin, analytes associated with CLIX score, analytes associated with HOMA (Homeostasis Model Assessment) score, analytes associated with IR score, anti-islet cell cytoplasmic (anti-ICA) auto-antibodies, C-peptide, cystatin C, D-mannose, estimated glomerular filtration rate (eGFR), F2-isoprostanes, ferritin, fibrinogen, free fatty acids, fructosamine, gamma-glutamic transferase (GGT), glucose, glutamic acid decarboxylase (anti-GAD) auto-antibodies, glycation gap (glycosylation gap), HDL, HDL-c, hemoglobin (Hb) A1c, homocysteine, insulin, intact pro-insulin, LDL, LDL-c, leptin, leptin/adiponectin ratio, linoleoyl glycerophosphocholine (L-GPC), lipoprotein-associated phospholipase A2 (Lp-PLA2), mannose binding lectin (MBL) activity, mannose, mannose-binding lectin (MBL) amount, myeloperoxidase, oleic acid (OA), pro-insulin, serum amylase, serum cholinesterase, serum creatinine, triglycerides, TSH, uric acid, vitamin B12, and any combination thereof. In an aspect, one or more of the cellular metabolites can be differentially present. In an aspect, a combination of cellular metabolites can be differentially present.

In an aspect, the one or more cellular metabolites can comprise uric acid, lactate, triglycerides, and any combination thereof. In an aspect, the one or more cellular metabolites can comprise serum cholinesterase, albumin, and any combination thereof.

In an aspect, a disclosed subject can have GSD I, GSD II, GSD III, GSD IV, GSD V, GSD VI, GSD VII, GSD IX, GSD XI, GSD XII, GSD XIII, GSD IX, GSD XIV, or GSD XV. In an aspect, a disclosed subject can have GSD III, GSD VI, GSD IX, or Fanconi-Bickel syndrome. In an aspect, a subject can have GSD Ia or GSD Ib.

In an aspect, a disclosed method can comprise administering to the subject one or more agents that modulate the level of one or more differentially present cellular metabolites. In an aspect, a disclosed agent can target G6PT dysfunction of G6PT deficiency. In an aspect, treating the subject cam comprise administering to the subject one or more agents that target renal sodium glucose co-transporter 2 (SGLT2), such as, for example, empagliflozin, canagliflozin, dapagliflozin, ipragliflozin, luseogliflozin, and Tofogliflozin. In an aspect, the SGLT2 inhibitor can be empagliflozin.

In an aspect of a disclosed method, treating a subject can comprise administering to the subject to the subject ERT, gene therapy, mRNA therapy, small molecule therapy, SRT, or any combination thereof. In an aspect of a disclosed method, treating a subject can comprise administering to the subject a combination of one or more agents with ERT, gene therapy, mRNA therapy, small molecule therapy, SRT, or any combination thereof.

In an aspect of a disclosed method, treating a subject can comprise implementing a change in the subject's dietary intake of carbohydrates. In an aspect, a change in a subject's dietary intake of carbohydrates can comprise adding carbohydrates to the subject's diet, or removing carbohydrates from the subject's diet, or changing the type of carbohydrates in the subject's diet, or changing the frequency of carbohydrates consumed by the subject.

In an aspect of a disclosed method, treating a subject can comprise administering cornstarch to the subject. Cornstarch is known to the art and can comprise uncooked cornstarch, modified cornstarch, a cornstarch derivative, a cornstarch alternative, or any combination thereof. In an aspect, cornstarch can comprise Glycosade . In an aspect, treating a subject can comprise administering glycoside to the subject.

In an aspect, a subject's treatment can comprise ingesting one or more anaplerotic agents (e.g., medium-even-chain triglycerides (MCT), triheptanoin, etc.). In an aspect, the one or more anaplerotic agents can reduce the amount of cornstarch and/or carbohydrates required by the subject to, for example, maintain metabolic control.

In an aspect, any step in the disclosed method can be repeated one or more times. In an aspect, one or more steps in the disclosed method can be repeated one or more times.

In an aspect, a step of a disclosed method can be modified. For example, in an aspect, modifying the method can comprise modifying or changing one or more features or aspects of one or more steps of a disclosed method. For example, in an aspect, a disclosed method can be altered by changing the amount of one or more of the disclosed agents, by changing the route or frequency of administration of one or more of the disclosed agents, or by changing the duration of time one or more of the disclosed agents are administered to a subject.

In an aspect of a disclosed method, gene therapy can comprise administering to the subject an isolated nucleic acid molecule encoding a protein that is deficient and/or impaired in the subject such as, for example, a deficient and/or impaired protein having a role in glycogenolysis and/or gluconeogenesis. In an aspect, a disclosed isolated nucleic acid can comprise the sequence for AGA, AGL, ALSOA, ARSA, ARSB, ASAH1, ATG5, ATG7, CGI58, CLCN5, CLN3, CLN5, CLN6, CLN8, CTNS, CTSA, CTSK, ENO3, EPM2A, FIG4, FUCA1, G6PC, G6PT (i.e., G6PT1, G6PT2, G6PT3), GAA, GALC, GALNS, GBA, GDE, GEB1, GLA, GLB1, GM2A, GNPTAB, GNPTG, GNS, GUSB, GYG1, GYS1, GYS2, HEXA, HEXB, HGSNAT, HYAL1, IDS, IDUA, LAMP2, LIPA, MAN2B1, MANBA, MCOLN1, mTORC1, NAGA, NAGLU, NEU1, NHLRC1, NPC1, NPC1, NPC2, OCRL, PNPLA2, PPT1, PSAP, PFKM, PHKA2, PHK, PHKG2, PHKA1, PGAM2, PRKAG2, SGSH, SLC17A5, SLC37A4, SLC38A9, SLC9A6, SMPD1, SUMF1, and TPP1.

In an aspect, gene therapy can comprise administering to the subject an isolated nucleic acid molecule encoding a native or recombinant protein, such as, for example, alpha-glucosidase (GAA), α-galactosidase A (GLA), acid β-glucosidase, α-L-iduronidase, iduronate 2-sulfatase, lysosomal acid lipase, liver glycogen synthase, muscle glycogen synthase, glucose-6-phosphatase α, glucose-6-phosphate transporter, α-glucosidase, glycogen debranching enzyme, glycogen branching enzyme, muscle glycogen phosphorylase, liver glycogen phosphorylase, muscle phosphofructose kinase, phosphorylase kinase (α2 subunit), phosphorylase kinase (β subunit), phosphorylase kinase (γ subunit), phosphorylase kinase (α1 subunit), muscle phosphoglycerate mutase, glucose transporter 2, aldolase A, γ2-subunit of AMP-activated protein kinase (AMPK), β-enolase, glycogenin-1, dystrophin, factor VIII, lysosomal-associated membrane protein 2, laforin, or malin, or a fragment thereof.

In an aspect, gene therapy can comprise using a disclosed viral vector or a disclosed non-viral vector (discussed supra). In an aspect, a disclosed viral vector can be a disclosed adeno-associated virus (AAV) vector. Naturally isolated serotypes as well as recombinant serotypes are known to the art and discussed supra. In an aspect, a disclosed vector or a disclosed isolated nucleic acid molecule can be delivered to the subject's liver, heart, skeletal muscle, smooth muscle, CNS, PNS, or a combination thereof. In an aspect, a disclosed vector can be delivered to the subject's liver. In an aspect, a disclosed nucleic acid molecule and/or a disclosed vector can comprise one or more disclosed regulatory sequences.

In an aspect, enzyme replacement therapy can comprise administering to the subject a native or recombinant protein, such as, for example, alpha-glucosidase (GAA), α-galactosidase A (GLA), acid β-glucosidase, α-L-iduronidase, iduronate 2-sulfatase, lysosomal acid lipase, liver glycogen synthase, muscle glycogen synthase, glucose-6-phosphatase α, glucose-6-phosphate transporter, α-glucosidase, glycogen debranching enzyme, glycogen branching enzyme, muscle glycogen phosphorylase, liver glycogen phosphorylase, muscle phosphofructose kinase, phosphorylase kinase (α2 subunit), phosphorylase kinase (β subunit), phosphorylase kinase (γ subunit), phosphorylase kinase (α1 subunit), muscle phosphoglycerate mutase, glucose transporter 2, aldolase A, γ2-subunit of AMP-activated protein kinase (AMPK), β-enolase, glycogenin-1, dystrophin, factor VIII, lysosomal-associated membrane protein 2, laforin, or malin, or a fragment thereof. In an aspect, a disclosed encoded protein can comprise one or more modifications to improve expression or efficacy of that protein in a subject, such as, for example, a human patient (e.g., codon optimization). Modifications to proteins and fragments thereof are known to the art.

In an aspect, a disclosed a method of determining the efficacy of a treatment can comprise administering one or more disclosed immune modulators. Immune modulators are known in the art and discussed supra. In an aspect, a disclosed immune modulator can comprise bortezomib, intravenous gamma globulin (IVIG), methotrexate, or any combination thereof. In an aspect, a disclosed immune modulator can comprise bortezomib alone or in combination with methotrexate.

In an aspect, a disclosed a method of determining the efficacy of a treatment can comprise administering a small molecule. In an aspect, a disclosed small molecule cannot traverse the blood-brain barrier in a meaningful quantity but is nonetheless therapeutic for a subject having a GSD. In an aspect, a disclosed small molecule can traverse the blood-brain barrier in a meaningful quantity to treat a GSD.

In an aspect, a disclosed a method of determining the efficacy of a treatment can comprise substrate reduction therapy (SRT). In an aspect, SRT can comprise administering one or more small molecule inhibitors of biosynthesis to reduce the concentration of accumulating substrate to a level where the residual degradative enzymes can maintain homeostasis.

Disclosed herein is a method of determining the efficacy of a treatment in a subject having a Glycogen Storage Disease (GSD), the method comprising administering a treatment to a subject having a GSD; obtaining a post-treatment biosample from the subject and determining the level of 1,5-anhydroglucitol (1,5-AG) in the biosample; and determining the efficacy of the treatment by comparing the level of 1,5-AG in the post-treatment biosample to the level of 1,5-AG in either a pre-treatment biosample or a reference biosample.

In an aspect, a subject can be an adult, a young adult, an adolescent, a child, or a newborn infant. In an aspect, a subject can be a male or a female.

In an aspect, a disclosed subject can have GSD I, GSD II, GSD III, GSD IV, GSD V, GSD VI, GSD VII, GSD IX, GSD XI, GSD XII, GSD XIII, GSD IX, GSD XIV, or GSD XV. In an aspect, a disclosed subject can have GSD III, GSD VI, GSD IX, or Fanconi-Bickel syndrome. In an aspect, a disclosed subject can have GSD Ia or GSD Ib.

In an aspect, a disclosed method can comprise obtaining a pre-treatment biosample from the subject and determining the level of 1,5-AG. A disclosed method can comprise obtaining a reference biosample from a subject not having a GSD or from an aggregate of subjects not having a GSD and determining the level of 1,5-AG.

In an aspect, a reference biosample can comprise a biosample from a subject that is an adult, a young adult, an adolescent, a child, or a newborn infant, or biosamples from subjects that are adults, young adults, adolescents, children, or newborn infants. In an aspect, a subject can be a male or a female.

In an aspect, a biosample can comprise EDTA, blood, serum, or plasma.

In an aspect, a disclosed treatment can be considered effective when the post-treatment level of 1,5-AG is lower than the subject's pre-treatment level of 1,5-AG or when the post-treatment level of 1,5-AG is within an acceptable range of the reference level of 1,5-AG.

In an aspect, a disclosed treatment can be considered ineffective when the post-treatment level of 1,5-AG is higher than the pre-treatment level of 1,5-AG, or when the post-treatment level of 1,5-AG is not within an acceptable range of the reference level of 1,5-AG.

In an aspect, a disclosed method can comprise determining the level of one or more cellular metabolites in the subject's post-treatment biosample and comparing the level of the one or more cellular metabolites in either the pre-treatment biosample or the reference biosample to identify one or more differentially present cellular metabolites.

In an aspect, one or more differentially present cellular metabolites can be associated with a Z-score of at least +1.5, at least +2, or more than +2, or one or more differentially present cellular metabolites can be associated with a Z-score of at least −1.5, at least −2, or more than −2. In an aspect, the differential level of one or more cellular metabolites can comprise at least a Cohen's d effect size of at least 0.2, at least 0.5, at least 0.8, or greater than 0.8. In an aspect, the differential level of one or more cellular metabolites can comprise at least a Cohen's d effect size of at least 1, at least 2, at least 3, or greater than 3.

In an aspect, determining the level of 1,5-AG in a biosample can comprise using mass spectrometry, ultra-performance liquid chromatography (UPLC), high-performance liquid chromatography (HPLC), mass spectrometry in conjunction with UPLC, LC/MS/MS, ELISA, Western blots, or any combination thereof. In an aspect, determining the level of the one or more cellular metabolites a biosample can comprise using mass spectrometry, ultra-performance liquid chromatography (UPLC), high-performance liquid chromatography (HPLC), mass spectrometry in conjunction with UPLC, LC/MS/MS, ELISA, Western blots, or any combination thereof. The skilled person can identify the method or technique that is most suitable for a given biosample. In an aspect, the same method or technique can be used for one or more biosamples or for all biosamples. In an aspect, a different method or technique can be used for one or more biosamples or for all biosamples.

In an aspect, the one or more cellular metabolites can comprise 25-hydroxyvitamin D, albumin, adiponectin, analytes associated with CLIX score, analytes associated with HOMA (Homeostasis Model Assessment) score, analytes associated with IR score, anti-islet cell cytoplasmic (anti-ICA) auto-antibodies, C-peptide, cystatin C, D-mannose, estimated glomerular filtration rate (eGFR), F2-isoprostanes, ferritin, fibrinogen, free fatty acids, fructosamine, gamma-glutamic transferase (GGT), glucose, glutamic acid decarboxylase (anti-GAD) auto-antibodies, glycation gap (glycosylation gap), HDL, HDL-c, hemoglobin (Hb) A1c, homocysteine, insulin, intact pro-insulin, LDL, LDL-c, leptin, leptin/adiponectin ratio, linoleoyl glycerophosphocholine (L-GPC), lipoprotein-associated phospholipase A2 (Lp-PLA2), mannose binding lectin (MBL) activity, mannose, mannose-binding lectin (MBL) amount, myeloperoxidase, oleic acid (OA), pro-insulin, serum amylase, serum cholinesterase, serum creatinine, triglycerides, TSH, uric acid, vitamin B12, and any combination thereof. In an aspect, one or more of the cellular metabolites can be differentially present. In an aspect, a combination of cellular metabolites can be differentially present.

In an aspect, the one or more cellular metabolites can comprise glucose, uric acid, lactate, triglycerides, and any combination thereof. In an aspect, the one or more cellular metabolites can comprise serum cholinesterase, albumin, and any combination thereof.

In an aspect, a disclosed treatment can comprise administering cornstarch to the subject. Cornstarch is known to the art and can comprise uncooked cornstarch, modified cornstarch, a cornstarch derivative, a cornstarch alternative, or any combination thereof. In an aspect, cornstarch can comprise Glycosade®. In an aspect, a disclosed treatment can comprise administering glycoside to the subject.

In an aspect, a subject's treatment can comprise ingesting one or more anaplerotic agents (e.g., medium-even-chain triglycerides (MCT), triheptanoin, etc.). In an aspect, the one or more anaplerotic agents can reduce the amount of cornstarch and/or carbohydrates required by the subject to, for example, maintain metabolic control.

In an aspect, a disclosed treatment can comprise implementing a change in the dietary intake of carbohydrates. In an aspect, a change in a subject's dietary intake of carbohydrates can comprise adding carbohydrates to the subject's diet, or removing carbohydrates from the subject's diet, or changing the type of carbohydrates in the subject's diet, or changing the frequency of carbohydrates consumed by the subject.

In an aspect, a disclosed treatment can comprise administering to the subject ERT, gene therapy, mRNA therapy, small molecule therapy, SRT, or any combination thereof. In an aspect of a disclosed method, treating a subject can comprise administering to the subject a combination of one or more agents with ERT, gene therapy, mRNA therapy, small molecule therapy, SRT, or any combination thereof.

In an aspect, a disclosed method can comprise administering to the subject one or more agents that modulate the level of one or more differentially present cellular metabolites. In an aspect, a disclosed agent can target G6PT dysfunction of G6PT deficiency. In an aspect, treating the subject cam comprise administering to the subject one or more agents that target renal sodium glucose co-transporter 2 (SGLT2), such as, for example, empagliflozin, canagliflozin, dapagliflozin, ipragliflozin, luseogliflozin, and Tofogliflozin. In an aspect, the SGLT2 inhibitor can be empagliflozin.

In an aspect, any step in the disclosed method can be repeated one or more times. In an aspect, one or more steps in the disclosed method can be repeated one or more times.

In an aspect, a step of a disclosed method can be modified. For example, in an aspect, modifying the method can comprise modifying or changing one or more features or aspects of one or more steps of a disclosed method. For example, in an aspect, a disclosed method can be altered by changing the amount of one or more of the disclosed agents, by changing the route or frequency of administration of one or more of the disclosed agents, or by changing the duration of time one or more of the disclosed agents are administered to a subject.

In an aspect of a disclosed method, gene therapy can comprise administering to the subject an isolated nucleic acid molecule encoding a protein that is deficient and/or impaired in the subject such as, for example, a deficient and/or impaired protein having a role in glycogenolysis and/or gluconeogenesis. In an aspect, a disclosed isolated nucleic acid can comprise the sequence for AGA, AGL, ALSOA, ARSA, ARSB, ASAH1, ATG5, ATG7, CGI58, CLCN5, CLN3, CLN5, CLN6, CLN8, CTNS, CTSA, CTSK, ENO3, EPM2A, FIG4, FUCA1, G6PC, G6PT (i.e., G6PT1, G6PT2, G6PT3), GAA, GALC, GALNS, GBA, GDE, GEB1, GLA, GLB1, GM2A, GNPTAB, GNPTG, GNS, GUSB, GYG1, GYS1, GYS2, HEXA, HEXB, HGSNAT, HYAL1, IDS, IDUA, LAMP2, LIPA, MAN2B1, MANBA, MCOLN1, mTORC1, NAGA, NAGLU, NEU1, NHLRC1, NPC1, NPC1, NPC2, OCRL, PNPLA2, PPT1, PSAP, PFKM, PHKA2, PHK, PHKG2, PHKA1, PGAM2, PRKAG2, SGSH, SLC17A5, SLC37A4, SLC38A9, SLC9A6, SMPD1, SUMF1, and TPP1.

In an aspect, gene therapy can comprise administering to the subject an isolated nucleic acid molecule encoding a native or recombinant protein, such as, for example, alpha-glucosidase (GAA), α-galactosidase A (GLA), acid β-glucosidase, α-L-iduronidase, iduronate 2-sulfatase, lysosomal acid lipase, liver glycogen synthase, muscle glycogen synthase, glucose-6-phosphatase α, glucose-6-phosphate transporter, α-glucosidase, glycogen debranching enzyme, glycogen branching enzyme, muscle glycogen phosphorylase, liver glycogen phosphorylase, muscle phosphofructose kinase, phosphorylase kinase (α2 subunit), phosphorylase kinase (β subunit), phosphorylase kinase (γ subunit), phosphorylase kinase (α1 subunit), muscle phosphoglycerate mutase, glucose transporter 2, aldolase A, γ2-subunit of AMP-activated protein kinase (AMPK), β-enolase, glycogenin-1, dystrophin, factor VIII, lysosomal-associated membrane protein 2, laforin, or malin, or a fragment thereof.

In an aspect, gene therapy can comprise using a disclosed viral vector or a disclosed non-viral vector (discussed supra). In an aspect, a disclosed viral vector can be a disclosed adeno-associated virus (AAV) vector. Naturally isolated serotypes as well as recombinant serotypes are known to the art and discussed supra. In an aspect, a disclosed vector or a disclosed isolated nucleic acid molecule can be delivered to the subject's liver, heart, skeletal muscle, smooth muscle, CNS, PNS, or a combination thereof. In an aspect, a disclosed vector can be delivered to the subject's liver. In an aspect, a disclosed nucleic acid molecule and/or a disclosed vector can comprise one or more disclosed regulatory sequences.

In an aspect, enzyme replacement therapy can comprise administering to the subject a native or recombinant protein, such as, for example, alpha-glucosidase (GAA), α-galactosidase A (GLA), acid β-glucosidase, α-L-iduronidase, iduronate 2-sulfatase, lysosomal acid lipase, liver glycogen synthase, muscle glycogen synthase, glucose-6-phosphatase α, glucose-6-phosphate transporter, α-glucosidase, glycogen debranching enzyme, glycogen branching enzyme, muscle glycogen phosphorylase, liver glycogen phosphorylase, muscle phosphofructose kinase, phosphorylase kinase (α2 subunit), phosphorylase kinase (β subunit), phosphorylase kinase (γ subunit), phosphorylase kinase (α1 subunit), muscle phosphoglycerate mutase, glucose transporter 2, aldolase A, γ2-subunit of AMP-activated protein kinase (AMPK), β-enolase, glycogenin-1, dystrophin, factor VIII, lysosomal-associated membrane protein 2, laforin, or malin, or a fragment thereof. In an aspect, a disclosed encoded protein can comprise one or more modifications to improve expression or efficacy of that protein in a subject, such as, for example, a human patient (e.g., codon optimization). Modifications to proteins and fragments thereof are known to the art.

In an aspect, a disclosed method of determining the efficacy of a treatment can comprise administering one or more disclosed immune modulators. Immune modulators are known in the art and discussed supra. In an aspect, a disclosed immune modulator can comprise bortezomib, intravenous gamma globulin (IVIG), methotrexate, or any combination thereof. In an aspect, a disclosed immune modulator can comprise bortezomib alone or in combination with methotrexate.

In an aspect, a disclosed method of determining the efficacy of a treatment can comprise administering a small molecule. In an aspect, a disclosed small molecule cannot traverse the blood-brain barrier in a meaningful quantity but is nonetheless therapeutic for a subject having a GSD. In an aspect, a disclosed small molecule can traverse the blood-brain barrier in a meaningful quantity to treat a GSD.

In an aspect, a disclosed method of determining the efficacy of a treatment can comprise SRT. In an aspect, SRT can comprise administering one or more small molecule inhibitors of biosynthesis to reduce the concentration of accumulating substrate to a level where the residual degradative enzymes can maintain homeostasis.

### G. Methods of Monitoring Metabolic Control Status

Disclosed herein is a method of monitoring metabolic control status in a subject having a Glycogen Storage Disease (GSD), the method comprising obtaining a biosample from a subject having a GSD and determining the level of 1,5-anhydroglucitol (1,5-AG) in the biosample; comparing the level of 1,5-AG in the biosample to the level of 1,5-AG in either a biosample previously obtained for the subject or a reference biosample; treating the subject; and repeating the obtaining and comparing steps to determine the effect of the treatment on the metabolic control status.

In an aspect, a subject can be an adult, a young adult, an adolescent, a child, or a newborn infant. In an aspect, a subject can be a male or a female.

In an aspect, a disclosed subject can have GSD I, GSD II, GSD III, GSD IV, GSD V, GSD VI, GSD VII, GSD IX, GSD XI, GSD XII, GSD XIII, GSD IX, GSD XIV, or GSD XV. In an aspect, a disclosed subject can have GSD III, GSD VI, GSD IX, or Fanconi-Bickel syndrome. In an aspect, a disclosed subject can have GSD Ia or GSD Ib.

In an aspect, a disclosed method can comprise obtaining a pre-treatment biosample from the subject and determining the level of 1,5-AG. A disclosed method can comprise obtaining a reference biosample from a subject not having a GSD or from an aggregate of subjects not having a GSD and determining the level of 1,5-AG.

In an aspect, when the level of 1,5-AG in the biosample is higher than the level of 1,5-AG in the previously obtained biosample or higher than the level of 1,5-AG in the reference biosample, then a disclosed method can comprise continuing to treat the subject for poor metabolic control.

In an aspect, wherein when the level of 1,5-AG in the biosample is higher than the level of 1,5-AG in the previously obtained biosample or higher than the level of 1,5-AG in the reference biosample, then a disclosed method can comprise modifying the treatment step.

In an aspect, a disclosed treatment can comprise administering cornstarch to the subject. Cornstarch is known to the art and can comprise uncooked cornstarch, modified cornstarch, a cornstarch derivative, a cornstarch alternative, or any combination thereof. In an aspect, cornstarch can comprise Glycosade®. In an aspect, a disclosed treatment can comprise administering glycoside to the subject.

In an aspect, a subject's treatment can comprise ingesting one or more anaplerotic agents (e.g., medium-even-chain triglycerides (MCT), triheptanoin, etc.). In an aspect, the one or more anaplerotic agents can reduce the amount of cornstarch and/or carbohydrates required by the subject to, for example, maintain metabolic control.

In an aspect, a disclosed treatment can comprise implementing a change in the dietary intake of carbohydrates. In an aspect, a change in a subject's dietary intake of carbohydrates can comprise adding carbohydrates to the subject's diet, or removing carbohydrates from the subject's diet, or changing the type of carbohydrates in the subject's diet, or changing the frequency of carbohydrates consumed by the subject.

In an aspect, a disclosed treatment can comprise enzyme replacement therapy, subject gene therapy, mRNA therapy, small molecule therapy, or any combination thereof.

In an aspect, a disclosed method can comprise determining the level of one or more cellular metabolites in the subject's post-treatment biosample and comparing the post-treatment level to the level of the one or more cellular metabolites in either the pre-treatment biosample or the reference biosample to identify one or more differentially present cellular metabolites.

In an aspect, determining the level of 1,5-AG in a biosample can comprise using mass spectrometry, ultra-performance liquid chromatography (UPLC), high-performance liquid chromatography (HPLC), mass spectrometry in conjunction with UPLC, LC/MS/MS, ELISA, Western blots, or any combination thereof. In an aspect, determining the level of the one or more cellular metabolites a biosample can comprise using mass spectrometry, ultra-performance liquid chromatography (UPLC), high-performance liquid chromatography (HPLC), mass spectrometry in conjunction with UPLC, LC/MS/MS, ELISA, Western blots, or any combination thereof. The skilled person can identify the method or technique that is most suitable for a given biosample. In an aspect, the same method or technique can be used for one or more biosamples or for all biosamples. In an aspect, a different method or technique can be used for one or more biosamples or for all biosamples.

In an aspect, a biosample can comprise EDTA, blood, serum, or plasma.

In an aspect, a disclosed method can comprise determining the level of one or more cellular metabolites in the subject's post-treatment biosample and comparing the level of the one or more cellular metabolites in either the pre-treatment biosample or the reference biosample to identify one or more differentially present cellular metabolites.

In an aspect, the one or more differentially present cellular metabolites can be associated with impaired glycogenolysis, impaired gluconeogenesis, impaired ketogenesis, or any combination thereof. In an aspect, the one or more differentially present cellular metabolites can be associated with lactic acidosis, hyperuricemia, hyperlipidemia, or any combination thereof. In an aspect, the one or more differentially present cellular metabolites can be associated with impaired glycogenolysis, impaired gluconeogenesis, impaired ketogenesis, lactic acidosis, hyperuricemia, hyperlipidemia, or any combination thereof.

In an aspect, the one or more cellular metabolites can comprise 25-hydroxyvitamin D, albumin, adiponectin, analytes associated with CLIX score, analytes associated with HOMA (Homeostasis Model Assessment) score, analytes associated with IR score, anti-islet cell cytoplasmic (anti-ICA) auto-antibodies, C-peptide, cystatin C, D-mannose, estimated glomerular filtration rate (eGFR), F2-isoprostanes, ferritin, fibrinogen, free fatty acids, fructosamine, gamma-glutamic transferase (GGT), glucose, glutamic acid decarboxylase (anti-GAD) auto-antibodies, glycation gap (glycosylation gap), HDL, HDL-c, hemoglobin (Hb) A1c, homocysteine, insulin, intact pro-insulin, LDL, LDL-c, leptin, leptin/adiponectin ratio, linoleoyl glycerophosphocholine (L-GPC), lipoprotein-associated phospholipase A2 (Lp-PLA2), mannose binding lectin (MBL) activity, mannose, mannose-binding lectin (MBL) amount, myeloperoxidase, oleic acid (OA), pro-insulin, serum amylase, serum cholinesterase, serum creatinine, triglycerides, TSH, uric acid, vitamin B12, and any combination thereof. In an aspect, one or more of the cellular metabolites can be differentially present. In an aspect, a combination of cellular metabolites can be differentially present.

In an aspect, the one or more cellular metabolites can comprise glucose, uric acid, lactate, triglycerides, and any combination thereof. In an aspect, the one or more cellular metabolites can comprise serum cholinesterase, albumin, and any combination thereof.

In an aspect, one or more differentially present cellular metabolites can be associated with a Z-score of at least +1.5, at least +2, or more than +2, or one or more differentially present cellular metabolites can be associated with a Z-score of at least −1.5, at least −2, or more than −2. In an aspect, the differential level of one or more cellular metabolites can comprise at least a Cohen's d effect size of at least 0.2, at least 0.5, at least 0.8, or greater than 0.8. In an aspect, the differential level of one or more cellular metabolites can comprise at least a Cohen's d effect size of at least 1, at least 2, at least 3, or greater than 3.

In an aspect, a disclosed method can comprise administering to the subject one or more agents that modulate the level of one or more differentially present cellular metabolites. In an aspect, a disclosed agent can target G6PT dysfunction of G6PT deficiency. In an aspect, treating the subject cam comprise administering to the subject one or more agents that target renal sodium glucose co-transporter 2 (SGLT2), such as, for example, empagliflozin, canagliflozin, dapagliflozin, ipragliflozin, luseogliflozin, and Tofogliflozin. In an aspect, the SGLT2 inhibitor can be empagliflozin.

In an aspect, a disclosed method can comprise administering to the subject to the subject ERT, gene therapy, mRNA therapy, small molecule therapy, SRT, or any combination thereof. In an aspect, a disclosed method can comprise administering to the subject a combination of one or more agents with ERT, gene therapy, mRNA therapy, small molecule therapy, SRT, or any combination thereof.

In an aspect, a disclosed method can comprise implementing a change in the subject's dietary intake of carbohydrates. In an aspect, a change in a subject's dietary intake of carbohydrates can comprise adding carbohydrates to the subject's diet, or removing carbohydrates from the subject's diet, or changing the type of carbohydrates in the subject's diet, or changing the frequency of carbohydrates consumed by the subject.

In an aspect, a disclosed method can comprise administering cornstarch to the subject. Cornstarch is known to the art and can comprise uncooked cornstarch, modified cornstarch, a cornstarch derivative, a cornstarch alternative, or any combination thereof. In an aspect, cornstarch can comprise Glycosade®. In an aspect, a disclosed method can comprise administering glycoside to the subject.

In an aspect, any step in the disclosed method can be repeated one or more times. In an aspect, one or more steps in the disclosed method can be repeated one or more times.

In an aspect, a step of a disclosed method can be modified. For example, in an aspect, modifying the method can comprise modifying or changing one or more features or aspects of one or more steps of a disclosed method. For example, in an aspect, a disclosed method can be altered by changing the amount of one or more of the disclosed agents, by changing the route or frequency of administration of one or more of the disclosed agents, or by changing the duration of time one or more of the disclosed agents are administered to a subject.

In an aspect of a disclosed method, gene therapy can comprise administering to the subject an isolated nucleic acid molecule encoding a protein that is deficient and/or impaired in the subject such as, for example, a deficient and/or impaired protein having a role in glycogenolysis and/or gluconeogenesis. In an aspect, a disclosed isolated nucleic acid can comprise the sequence for AGA, AGL, ALSOA, ARSA, ARSB, ASAH1, ATG5, ATG7, CGI58, CLCN5, CLN3, CLN5, CLN6, CLN8, CTNS, CTSA, CTSK, ENO3, EPM2A, FIG4, FUCA1, G6PC, G6PT (i.e., G6PT1, G6PT2, G6PT3), GAA, GALC, GALNS, GBA, GDE, GEB1, GLA, GLB1, GM2A, GNPTAB, GNPTG, GNS, GUSB, GYG1, GYS1, GYS2, HEXA, HEXB, HGSNAT, HYAL1, IDS, IDUA, LAMP2, LIPA, MAN2B1, MANBA, MCOLN1, mTORC1, NAGA, NAGLU, NEU1, NHLRC1, NPC1, NPC1, NPC2, OCRL, PNPLA2, PPT1, PSAP, PFKM, PHKA2, PHK, PHKG2, PHKA1, PGAM2, PRKAG2, SGSH, SLC17A5, SLC37A4, SLC38A9, SLC9A6, SMPD1, SUMF1, and TPP1.

In an aspect, gene therapy can comprise administering to the subject an isolated nucleic acid molecule encoding a native or recombinant protein, such as, for example, alpha-glucosidase (GAA), α-galactosidase A (GLA), acid β-glucosidase, α-L-iduronidase, iduronate 2-sulfatase, lysosomal acid lipase, liver glycogen synthase, muscle glycogen synthase, glucose-6-phosphatase α, glucose-6-phosphate transporter, α-glucosidase, glycogen debranching enzyme, glycogen branching enzyme, muscle glycogen phosphorylase, liver glycogen phosphorylase, muscle phosphofructose kinase, phosphorylase kinase (α2 subunit), phosphorylase kinase (β subunit), phosphorylase kinase (γ subunit), phosphorylase kinase (α1 subunit), muscle phosphoglycerate mutase, glucose transporter 2, aldolase A, γ2-subunit of AMP-activated protein kinase (AMPK), β-enolase, glycogenin-1, dystrophin, factor VIII, lysosomal-associated membrane protein 2, laforin, or malin, or a fragment thereof.

In an aspect, gene therapy can comprise using a disclosed viral vector or a disclosed non-viral vector (discussed supra). In an aspect, a disclosed viral vector can be a disclosed adeno-associated virus (AAV) vector. Naturally isolated serotypes as well as recombinant serotypes are known to the art and discussed supra. In an aspect, a disclosed vector or a disclosed isolated nucleic acid molecule can be delivered to the subject's liver, heart, skeletal muscle, smooth muscle, CNS, PNS, or a combination thereof. In an aspect, a disclosed vector can be delivered to the subject's liver. In an aspect, a disclosed nucleic acid molecule and/or a disclosed vector can comprise one or more disclosed regulatory sequences.

In an aspect, enzyme replacement therapy can comprise administering to the subject a native or recombinant protein, such as, for example, alpha-glucosidase (GAA), α-galactosidase A (GLA), acid β-glucosidase, α-L-iduronidase, iduronate 2-sulfatase, lysosomal acid lipase, liver glycogen synthase, muscle glycogen synthase, glucose-6-phosphatase α, glucose-6-phosphate transporter, α-glucosidase, glycogen debranching enzyme, glycogen branching enzyme, muscle glycogen phosphorylase, liver glycogen phosphorylase, muscle phosphofructose kinase, phosphorylase kinase (α2 subunit), phosphorylase kinase (β subunit), phosphorylase kinase (γ subunit), phosphorylase kinase (α1 subunit), muscle phosphoglycerate mutase, glucose transporter 2, aldolase A, γ2-subunit of AMP-activated protein kinase (AMPK), β-enolase, glycogenin-1, dystrophin, factor VIII, lysosomal-associated membrane protein 2, laforin, or malin, or a fragment thereof. In an aspect, a disclosed encoded protein can comprise one or more modifications to improve expression or efficacy of that protein in a subject, such as, for example, a human patient (e.g., codon optimization). Modifications to proteins and fragments thereof are known to the art.

In an aspect, a disclosed method of monitoring metabolic control status can comprise administering one or more disclosed immune modulators. Immune modulators are known in the art and discussed supra. In an aspect, a disclosed immune modulator can comprise bortezomib, intravenous gamma globulin (IVIG), methotrexate, or any combination thereof. In an aspect, a disclosed immune modulator can comprise bortezomib alone or in combination with methotrexate.

In an aspect, a disclosed method of monitoring metabolic control status can comprise administering a small molecule. In an aspect, a disclosed small molecule cannot traverse the blood-brain barrier in a meaningful quantity but is nonetheless therapeutic for a subject having a GSD. In an aspect, a disclosed small molecule can traverse the blood-brain barrier in a meaningful quantity to treat a GSD.

In an aspect, a disclosed method of monitoring metabolic control status can comprise SRT. In an aspect, SRT can comprise administering one or more small molecule inhibitors of biosynthesis to reduce the concentration of accumulating substrate to a level where the residual degradative enzymes can maintain homeostasis.

### H. Methods of Determining the Effect of Regulating Carbohydrate Intake

Disclosed herein is a method of determining the effect of regulating carbohydrate intake in a subject, the method comprising implementing a change in the dietary intake of carbohydrates in a subject having a Glycogen Storage Disease (GSD); obtaining a biosample from the subject; determining the level of 1,5-anhydroglucitol (1,5-AG) in the biosample; and comparing the level of 1,5-AG in the biosample to the level of 1,5-AG in either a pre-dietary change biosample or a reference biosample.

In an aspect, a disclosed method can comprise obtaining a biosample from the subject prior to implementing a change in the dietary intake of carbohydrates and determining the level of 1,5-AG.

In an aspect, a subject's treatment can comprise ingesting one or more anaplerotic agents (e.g., medium-even-chain triglycerides (MCT), triheptanoin, etc.). In an aspect, a disclosed method of determining the effect of regulating carbohydrate intake comprises determining whether the one or more anaplerotic agents ingested by the subject can decrease the amount of uncooked cornstarch, modified cornstarch, a cornstarch derivative, a cornstarch alternative, or combination thereof required to achieve metabolic control in the subject. In an aspect, the one or more anaplerotic agents can reduce the amount of cornstarch required to achieve metabolic control in the subject. As known to the art, triheptanoin is an anaplerotic medium chain triglyceride that provides both even and odd chain substrates to the TCA cycle, restoring its balance and improving ATP generation in experimental systems. As known to the art, medium-chain triglycerides (MCTs) contain fatty acids that have a chain length of 6-12 carbon atoms. They include caproic acid (C6), caprylic acid (C8), capric acid (C10), and lauric acid (C12).

In an aspect, implementing a change in the dietary intake of carbohydrates can comprise adding carbohydrates to the subject's diet, or removing carbohydrates from the subject's diet, or changing the type of carbohydrates in the subject's diet, or changing the frequency of carbohydrates consumed by the subject. In an aspect, the one or more anaplerotic agents can reduce the amount of carbohydrates required to achieve metabolic control in the subject.

In an aspect, a biosample can comprise EDTA, blood, serum, or plasma.

In an aspect, a disclosed method can comprise obtaining a biosample from the subject not having a GSD and determining the level of 1,5-AG. In an aspect, a disclosed method can comprise obtaining a biosample from subjects not having a GSD and determining the level of 1,5-AG.

In an aspect, when the level of 1,5-AG in the subject's biosample is higher than the level of 1,5-AG in the pre-dietary change biosample or higher than the level of 1,5-AG in the reference biosample, then a disclosed method can comprise treating the subject.

In an aspect, the higher level of 1,5-AG in the subject having a GSD can be associated with a Z-score of at least +1.5, at least +2, or more than +2.

In an aspect, a disclosed method can comprise administering cornstarch to the subject. Cornstarch is known to the art and can comprise uncooked cornstarch, modified cornstarch, a cornstarch derivative, a cornstarch alternative, or any combination thereof. In an aspect, cornstarch can comprise Glycosade®. In an aspect, a disclosed method can comprise administering glycoside to the subject.

In an aspect, a subject can be an adult, a young adult, an adolescent, a child, or a newborn infant. In an aspect, a subject can be a male or a female.

In an aspect, a disclosed subject can have GSD I, GSD II, GSD III, GSD IV, GSD V, GSD VI, GSD VII, GSD IX, GSD XI, GSD XII, GSD XIII, GSD IX, GSD XIV, or GSD XV. In an aspect, a disclosed subject can have GSD III, GSD VI, GSD IX, or Fanconi-Bickel syndrome. In an aspect, a disclosed

In an aspect, wherein when the level of 1,5-AG in the subject's biosample is higher than the level of 1,5-AG in the pre-dietary change biosample or higher than the level of 1,5-AG in the reference biosample, then a disclosed method can comprise modifying the dietary change.

In an aspect, treating the subject can comprise administering cornstarch to the subject. Cornstarch is known to the art and can comprise uncooked cornstarch, modified cornstarch, a cornstarch derivative, a cornstarch alternative, or any combination thereof. In an aspect, cornstarch can comprise Glycosade®. In an aspect, treating the subject can comprise administering glycoside to the subject.

In an aspect, treating a subject can comprise ERT, gene therapy, mRNA therapy, small molecule therapy, SRT, or any combination thereof. In an aspect, a disclosed method can comprise administering to the subject a combination of one or more agents with ERT, gene therapy, mRNA therapy, small molecule therapy, SRT, or any combination thereof.

In an aspect, a disclosed method can comprise determining the level of one or more cellular metabolites in the subject's post-dietary change biosample and comparing the post-dietary change level to the level of the one or more cellular metabolites in either the pre-dietary change biosample or the reference biosample to identify one or more differentially present cellular metabolites.

In an aspect, determining the level of 1,5-AG in a biosample can comprise using mass spectrometry, ultra-performance liquid chromatography (UPLC), high-performance liquid chromatography (HPLC), mass spectrometry in conjunction with UPLC, LC/MS/MS, ELISA, Western blots, or any combination thereof. In an aspect, determining the level of the one or more cellular metabolites a biosample can comprise using mass spectrometry, ultra-performance liquid chromatography (UPLC), high-performance liquid chromatography (HPLC), mass spectrometry in conjunction with UPLC, LC/MS/MS, ELISA, Western blots, or any combination thereof. The skilled person can identify the method or technique that is most suitable for a given biosample. In an aspect, the same method or technique can be used for one or more biosamples or for all biosamples. In an aspect, a different method or technique can be used for one or more biosamples or for all biosamples.

In an aspect, the one or more differentially present cellular metabolites can be associated with impaired glycogenolysis, impaired gluconeogenesis, impaired ketogenesis, or any combination thereof. In an aspect, the one or more differentially present cellular metabolites can be associated with lactic acidosis, hyperuricemia, hyperlipidemia, or any combination thereof. In an aspect, the one or more differentially present cellular metabolites can be associated with impaired glycogenolysis, impaired gluconeogenesis, impaired ketogenesis, lactic acidosis, hyperuricemia, hyperlipidemia, or any combination thereof.

In an aspect, the one or more cellular metabolites can comprise 25-hydroxyvitamin D, albumin, adiponectin, analytes associated with CLIX score, analytes associated with HOMA (Homeostasis Model Assessment) score, analytes associated with IR score, anti-islet cell cytoplasmic (anti-ICA) auto-antibodies, C-peptide, cystatin C, D-mannose, estimated glomerular filtration rate (eGFR), F2-isoprostanes, ferritin, fibrinogen, free fatty acids, fructosamine, gamma-glutamic transferase (GGT), glucose, glutamic acid decarboxylase (anti-GAD) auto-antibodies, glycation gap (glycosylation gap), HDL, HDL-c, hemoglobin (Hb) A1c, homocysteine, insulin, intact pro-insulin, LDL, LDL-c, leptin, leptin/adiponectin ratio, linoleoyl glycerophosphocholine (L-GPC), lipoprotein-associated phospholipase A2 (Lp-PLA2), mannose binding lectin (MBL) activity, mannose, mannose-binding lectin (MBL) amount, myeloperoxidase, oleic acid (OA), pro-insulin, serum amylase, serum cholinesterase, serum creatinine, triglycerides, TSH, uric acid, vitamin B12, and any combination thereof. In an aspect, one or more of the cellular metabolites can be differentially present. In an aspect, a combination of cellular metabolites can be differentially present.

In an aspect, the one or more cellular metabolites can comprise glucose, uric acid, lactate, triglycerides, and any combination thereof. In an aspect, the one or more cellular metabolites can comprise serum cholinesterase, albumin, and any combination thereof.

In an aspect, one or more differentially present cellular metabolites can be associated with a Z-score of at least +1.5, at least +2, or more than +2, or one or more differentially present cellular metabolites can be associated with a Z-score of at least −1.5, at least −2, or more than −2. In an aspect, the differential level of one or more cellular metabolites can comprise at least a Cohen's d effect size of at least 0.2, at least 0.5, at least 0.8, or greater than 0.8. In an aspect, the differential level of one or more cellular metabolites can comprise at least a Cohen's d effect size of at least 1, at least 2, at least 3, or greater than 3.

In an aspect, a disclosed method can comprise administering to the subject one or more agents that modulate the level of one or more differentially present cellular metabolites. In an aspect, a disclosed agent can target G6PT dysfunction of G6PT deficiency. In an aspect, treating the subject cam comprise administering to the subject one or more agents that target renal sodium glucose co-transporter 2 (SGLT2), such as, for example, empagliflozin, canagliflozin, dapagliflozin, ipragliflozin, luseogliflozin, and Tofogliflozin. In an aspect, the SGLT2 inhibitor can be empagliflozin.

In an aspect, any step in the disclosed method can be repeated one or more times. In an aspect, one or more steps in the disclosed method can be repeated one or more times.

In an aspect, a step of a disclosed method can be modified. For example, in an aspect, modifying the method can comprise modifying or changing one or more features or aspects of one or more steps of a disclosed method. For example, in an aspect, a disclosed method can be altered by changing the amount of one or more of the disclosed agents, by changing the route or frequency of administration of one or more of the disclosed agents, or by changing the duration of time one or more of the disclosed agents are administered to a subject.

In an aspect of a disclosed method, gene therapy can comprise administering to the subject an isolated nucleic acid molecule encoding a protein that is deficient and/or impaired in the subject such as, for example, a deficient and/or impaired protein having a role in glycogenolysis and/or gluconeogenesis. In an aspect, a disclosed isolated nucleic acid can comprise the sequence for AGA, AGL, ALSOA, ARSA, ARSB, ASAH1, ATG5, ATG7, CGI58, CLCN5, CLN3, CLN5, CLN6, CLN8, CTNS, CTSA, CTSK, ENO3, EPM2A, FIG4, FUCA1, G6PC, G6PT (i.e., G6PT1, G6PT2, G6PT3), GAA, GALC, GALNS, GBA, GDE, GEB1, GLA, GLB1, GM2A, GNPTAB, GNPTG, GNS, GUSB, GYG1, GYS1, GYS2, HEXA, HEXB, HGSNAT, HYAL1, IDS, IDUA, LAMP2, LIPA, MAN2B1, MANBA, MCOLN1, mTORC1, NAGA, NAGLU, NEU1, NHLRC1, NPC1, NPC1, NPC2, OCRL, PNPLA2, PPT1, PSAP, PFKM, PHKA2, PHK, PHKG2, PHKA1, PGAM2, PRKAG2, SGSH, SLC17A5, SLC37A4, SLC38A9, SLC9A6, SMPD1, SUMF1, and TPP1.

In an aspect, gene therapy can comprise administering to the subject an isolated nucleic acid molecule encoding a native or recombinant protein, such as, for example, alpha-glucosidase (GAA), α-galactosidase A (GLA), acid β-glucosidase, α-L-iduronidase, iduronate 2-sulfatase, lysosomal acid lipase, liver glycogen synthase, muscle glycogen synthase, glucose-6-phosphatase α, glucose-6-phosphate transporter, α-glucosidase, glycogen debranching enzyme, glycogen branching enzyme, muscle glycogen phosphorylase, liver glycogen phosphorylase, muscle phosphofructose kinase, phosphorylase kinase (α2 subunit), phosphorylase kinase (β subunit), phosphorylase kinase (γ subunit), phosphorylase kinase (α1 subunit), muscle phosphoglycerate mutase, glucose transporter 2, aldolase A, γ2-subunit of AMP-activated protein kinase (AMPK), β-enolase, glycogenin-1, dystrophin, factor VIII, lysosomal-associated membrane protein 2, laforin, or malin, or a fragment thereof.

In an aspect, gene therapy can comprise using a disclosed viral vector or a disclosed non-viral vector (discussed supra). In an aspect, a disclosed viral vector can be a disclosed adeno-associated virus (AAV) vector. Naturally isolated serotypes as well as recombinant serotypes are known to the art and discussed supra. In an aspect, a disclosed vector or a disclosed isolated nucleic acid molecule can be delivered to the subject's liver, heart, skeletal muscle, smooth muscle, CNS, PNS, or a combination thereof. In an aspect, a disclosed vector can be delivered to the subject's liver. In an aspect, a disclosed nucleic acid molecule and/or a disclosed vector can comprise one or more disclosed regulatory sequences.

In an aspect, enzyme replacement therapy can comprise administering to the subject a native or recombinant protein, such as, for example, alpha-glucosidase (GAA), α-galactosidase A (GLA), acid β-glucosidase, α-L-iduronidase, iduronate 2-sulfatase, lysosomal acid lipase, liver glycogen synthase, muscle glycogen synthase, glucose-6-phosphatase α, glucose-6-phosphate transporter, α-glucosidase, glycogen debranching enzyme, glycogen branching enzyme, muscle glycogen phosphorylase, liver glycogen phosphorylase, muscle phosphofructose kinase, phosphorylase kinase (α2 subunit), phosphorylase kinase (β subunit), phosphorylase kinase (γ subunit), phosphorylase kinase (α1 subunit), muscle phosphoglycerate mutase, glucose transporter 2, aldolase A, γ2-subunit of AMP-activated protein kinase (AMPK), β-enolase, glycogenin-1, dystrophin, factor VIII, lysosomal-associated membrane protein 2, laforin, or malin, or a fragment thereof. In an aspect, a disclosed encoded protein can comprise one or more modifications to improve expression or efficacy of that protein in a subject, such as, for example, a human patient (e.g., codon optimization). Modifications to proteins and fragments thereof are known to the art.

In an aspect, a disclosed method of determining the effect of regulating carbohydrate intake can comprise administering one or more disclosed immune modulators. Immune modulators are known in the art and discussed supra. In an aspect, a disclosed immune modulator can comprise bortezomib, intravenous gamma globulin (IVIG), methotrexate, or any combination thereof. In an aspect, a disclosed immune modulator can comprise bortezomib alone or in combination with methotrexate.

In an aspect, a disclosed method of determining the effect of regulating carbohydrate intake can comprise administering a small molecule. In an aspect, a disclosed small molecule cannot traverse the blood-brain barrier in a meaningful quantity but is nonetheless therapeutic for a subject having a GSD. In an aspect, a disclosed small molecule can traverse the blood-brain barrier in a meaningful quantity to treat a GSD.

In an aspect, a disclosed method of determining the effect of regulating carbohydrate intake can comprise SRT. In an aspect, SRT can comprise administering one or more small molecule inhibitors of biosynthesis to reduce the concentration of accumulating substrate to a level where the residual degradative enzymes can maintain homeostasis.

### I. Kits

Disclosed herein is a kit comprising one or more disclosed compositions for use in one or more disclosed methods. For example, disclosed herein is a kit comprising one or more disclosed compositions, pharmaceutical formulations, agents or therapeutic agents, immune modulators, proteasome inhibitors, small molecules, endonucleases, oligonucleotides, and/or the RNA therapeutic agents. In an aspect, a disclosed kit can comprise at least two components constituting the kit. Together, the components constitute a functional unit for a given purpose (such as, for example, detecting a GSD biomarker, determining the level of a GSD biomarker, comparing the level of a GSD biomarker to a reference biosample, repeating one or more steps of a disclosed method, or any combination thereof). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed composition, pharmaceutical formulation, agent or therapeutic agent, immune modulator proteasome inhibitor, small molecule, endonuclease, oligonucleotide, RNA therapeutic agent, or any combination thereof, and a label or package insert with instructions for use. In an aspect, suitable containers include, for example, bottles, vials, syringes, blister pack, etc. The containers can be formed from a variety of materials such as glass or plastic. The container can hold a disclosed composition, pharmaceutical formulation, agent or therapeutic agent, immune modulator, proteasome inhibitor, small molecule, endonuclease, oligonucleotide, RNA therapeutic agent, or any combination thereof, and can have a sterile access port (for example the container may be an intravenous solution bag or a vial having a stopper pierceable by a hypodermic injection needle). The label or package insert can indicate that a disclosed composition, pharmaceutical formulation, agent or therapeutic agent, immune modulator proteasome inhibitor, small molecule, endonuclease, oligonucleotide, and/or the RNA therapeutic agent, or any combination thereof can be used for treating, preventing, inhibiting, and/or ameliorating a GSD or complications and/or symptoms associated with a GSD (such as, for example, GSD Ia or GSD Ib). A kit can comprise additional components necessary for administration such as, for example, other buffers, diluents, filters, needles, and syringes for use in a disclosed method. In an aspect, a disclosed kit can comprise reagents for use in detecting 1,5-AG in one or more biosample.

### V. EXAMPLES

Glycogen refers to a branched polysaccharide with a molecular weight of 9-10 million Daltons. The average glycogen molecule contains about 55,000 glucosyl residues linked by α-1,4 (92%) and α-1,6 (8%) glycosidic bonds. Glycogen synthesis is catalyzed by the actions of 3 enzymes: (a) glycogenin (GYG), the initiating enzyme that starts a primer of glucose chain attached to itself; (b) glycogen synthase (GYS), which strings glucose to extend linear chains; and (c) glycogen-branching enzyme (GBE), which attaches a short new branch to a linear chain.

Glycogen storage disease type I (GSD I), also known as Von Gierke disease, is an inherited disorder caused by deficiencies of specific enzymes in the glycogen metabolism pathway. It was first described by Von Gierke in 1929 who reported excessive hepatic and renal glycogen in the autopsy reports of 2 children. It comprises 2 major subtypes—GSD Ia (MIM232200) and GSD Ib (MIM232220). (Kishnani et al. (2014) Genet. Med. 16(11):e1). The incidence of GSD I in the overall population is 1/100,000 with GSD Ia and Ib prevalent in 80% and 20%, respectively.

GSD Ia results from mutations in the G6PC gene on chromosome 17q21 that encodes for the G6Pase-a catalytic subunit. GSD Ib results from mutations in the SLC37A4 gene on chromosome 11q23.3. (Kishnani et al., 2014). In GSD Ia, there is a deficiency of enzyme glucose-6-phosphatase (G6Pase), which cleaves glycogen to glucose thus leading to hypoglycemia and lactic acidosis. (Wortmann, et al. (2020) Blood. 136(9):1033-1043).

The enzyme G6Pase is primarily expressed in the liver, kidney, and intestine. It has its active site on the luminal side of the endoplasmic reticulum (ER). Glucose-6-phosphate translocase is responsible for translocating Glucose-6-phosphate (G6P) from the cytoplasm into the ER lumen. The complex of G6Pase and G6PT catalyzes the final step of both glycogenolysis and gluconeogenesis for glucose production. Deficiency of either causes an accumulation of glycogen and fat in the liver, kidney, and intestinal mucosa. Patients with GSD lb have normal G6Pase enzyme activity but have a deficiency of the transporter enzyme, glucose-6-phosphate translocase (G6PT). (Kishnani et al. 2014). Patients present with manifestations of hypoglycemia and metabolic acidosis typically around 3 to 4 months of age. In patients suspected of having the disease, genetic testing is the investigation of choice to confirm the diagnosis. Dietary treatment prevents hypoglycemia and improves the life expectancy of patients. However, to prevent long-term complications such as hepatic adenomas and renal failure, animal models of GSD I are being developed to study the disease more closely and develop new treatment strategies such as gene therapy. (Al-Jobori et al. (2017) Diabetes. 66(7): 1999-2006).

Fasting hypoglycemia in GSD I is associated with impaired glycogenolysis, gluconeogenesis, and ketogenesis. Other metabolic derangements include, but are not limited to, lactic acidosis, hyperuricemia, and hyperlipidemia. The clinical manifestations of GSD I include stunted growth, hepatomegaly, nephromegaly, proteinuria, renal tubular acidosis, and combinations thereof. Late complications include renal failure and stones, polycystic ovarian syndrome, and hepatic adenomas with a risk of malignant transformation. Patients with GSD I have similar clinical manifestations; however, GSD Ib is associated with neutropenia, recurrent infections, and inflammatory bowel disease. (Kishnani et al., 2014). The latter is also observed in some patients with GSD Ia.

Currently, the standard of care therapy is nutritional intervention. Nutritional intervention requires rigorously scheduled frequent feeds/meals/snacks and the consumption of uncooked cornstarch between meals to maintain normoglycemia throughout the day and night. Meals and snacks must limit fructose, sucrose, and galactose and must include complex carbohydrates to prevent excursions in glucose levels because of an insulin response. Monitoring the metabolic status of patients with GSD I is essential to prevent complications (Kishnani et al., 2014). Monitoring the amount of carbohydrate intake in patients with GSD I is important because a subject's consumption of too much or too little is hazardous. When a subject's carbohydrate intake is not balanced, it can lead to increased glycogen and fat storage in the liver and kidneys, hypoglycemia, and late complications. Current clinical monitoring depends on daily frequent blood glucose checks throughout the day and night using a glucometer or continuous glucose monitor (if possible) as well as lactate, uric acid, and triglyceride serial evaluations during clinic visits. Additionally, in subjects having GSD Ib, it is necessary to monitor white blood cell and absolute neutrophil counts as well as inflammatory markers, which are often elevated.

### Example 1

### Review of 1,5-Anhydroglucitol-6-Phosphate (1,5-AG6P) Pathway

The structural analog of glucose 6 phosphate is 1,5-anhydroglucitol-6-phosphate (1,5-AG6P). Normally, 1,5-anhydroglucitol (1,5-AG) (PubChem 64960 or KEGG C07326) is phosphorylated to 1,5-anhydroglucitol-6-phosphate (1,5-AG6P) by hexokinases and ADP-dependent glucokinase (ADPGK) present in neutrophils. G6PT transports both glucose-6-phosphate and its structural analogue 1,5-anhydroglucitol-6-phosphate (1,5-AG6P) into the endoplasmic reticulum, where they are dephosphorylated. This transport prevents the accumulation of 1,5-AG6P in neutrophils. Instead, in subjects with GSD Ib (who have defective G6PT activity), 1,5-AG6P accumulates in neutrophils in toxic levels. (Wortmann et al., 2020). This toxic accumulation causes inhibition of the side chain hexokinases and intracellular depletion of the glucose 6 phosphate pool that is crucial for the function and survival of neutrophils. The reduction in the toxic accumulation of 1,5-AG6P in subjects with GSD Ib restores the functions of hexokinases, and the effects of the glucose 6 phosphate pool and other downstream metabolites leading to improved glycolysis, white blood cell respiratory burst function, and protein glycosylation. (Wortmann et al., 2020).

1,5 Anhydroglucitol (1,5-AG) is a polyol monosaccharide with a structure like D-Glucose. It has a 1-deoxy glucopyranose ring structure without a hydroxyl group at C1, making it metabolically stable. Normally, the origin of 1,5-AG is mostly from foods, with a limited amount coming from endogenous de novo synthesis. Free 1,5-AG is available in all organs and tissues, with the total amount being higher than that in plasma. (Yamanouchi et al. (1994) Diabetes Res. Clin. Pract. 24 Suppl:S261-268). About 99.9% of the excreted amount in urine is then reabsorbed by the kidneys. Reabsorption occurs at a specific sodium glucose active cotransporter (SGLT4). 1,5-AG has a half-life of 1-2 weeks in blood, the level of which is maintained by the balance between the dietary intake oral and urinary excretion. (Dungan (2008) Expert Rev. Mol. Diagn. 8(1):9-19; Kilpatrick et al. (1999) Diabet. Med. 16(6):496-499). High urinary glucose excretion competes with the tubular reabsorption of 1,5-AG, leading to its loss in urine and a decrease in 1,5-AG levels in serum compared to normal controls. 1,5-AG levels may be altered by renal failure (Yamanouchi et al., 1994; Dungan 2008); Kilpatrick et al., 1999) or liver cirrhosis (Koga et al., (2011) Ann Clin. Biochem. 48(Pt. 2); 121-125), which should be taken into consideration when evaluating levels in patients with restricted kidney and/or liver functions.

These data represent the first report of 1,5-AG as a biomarker for detecting and diagnosing a GSD as well as monitoring a GSD subject's overall metabolic control and responsiveness to treatment including dietary intervention.

### Example 2

### The Level of 1,5-AG Data is Elevated in GSD Subjects

The level of 1,5-AG was determined in 9 subjects having GSD Ia. Here, the reference range of 1,5-AG in normal, non-GSD Ia adults is 10.7-32.0.

| Subject # | Level of 1,5-AG | Cornstarch Dose |
|---|---|---|
| 1 | 58.3 | high |
| 2 | 33.1 | |
| 3 | 26.9 | |
| 4 | 35.0 | |
| 5 | 26.2 | None post-liver transplant |
| 6 | 22.2 | |
| 7 | 34.0 | |
| 8 | 63.1 | high |
| 9 | 62.1 | high |

The level of 1,5-AG was determined in 6 subjects having GSD Ib. Here, the reference range of 1,5-AG in normal, non-GSD Ib adults is 10.7-32.0.

| Subject # | Level of 1,5-AG | After Jardiance Treatment |
|---|---|---|
| 1 | 50.3 | |
| 2 | 46.3 | |
| 3 | 36.8 | |
| 4 | 59 | |
| 5 | 78 | 20.7, then 7.8 |
| 6 | 65.1 | 33.1 |

The level of 1,5-AG was determined in 3 subjects having GSD Ma. Here, the reference range of 1,5-AG in normal, non-GSD Ma adults is 10.7-32.0.

| Subject # | Level of 1,5-AG |
|---|---|
| 1 | 12.3 |
| 2 | 37.5 |
| 3 | 21.9 |

The level of 1,5-AG was determined in 1 subject having GSD VI. Here, the reference range of 1,5-AG in normal, non-GSD VI adults is 10.7-32.0.

| Subject # | Level of 1,5-AG |
|---|---|
| 1 | 34.8 |

The level of 1,5-AG was determined in 4 subjects having GSD IX. Here, the reference range of 1,5-AG in normal, non-GSD IX adults is 10.7-32.0.

| Subject # | GSD IX Subtype | Level of 1,5-AG | Cornstarch Dose |
|---|---|---|---|
| 1 | PHKG2 | 18.9 | |
| 2 | PHKG2 | 26.3 | |
| 3 | PHKB | 46.2 | high |
| 4 | PHKB | 38.2 | high |

Based on these data, the Z-score (that is, the number of standard deviations from the mean of the normal or reference level of 1,5-AG) associated with the elevated level of 1,5-AG in 5 subjects having GSD Ia was generated. Both GSD Ia subjects having poor metabolic control status had a Z-score greater than 2. Here, the Z-score reference range was as follows: the 2.5^{th} percentile was −1.9867 while the 97.5^{th} percentile was 1.6173.

| Subject # | Level of 1,5-AG | Metabolic Control |
|---|---|---|
| 1 (122377) | 1.8520 | |
| 2 (100196) | 1.9538 | |
| 3 (1100205) | 2.1584 | Poor |
| 4 (1100245) | 2.3047 | Poor |
| 5 (100270) | 1.9737 | |

These data show for the first time that 1,5-AG can be used as a biomarker for detecting and diagnosing a GSD such as GSD I. These data also show for the first time that 1,5-AG can be used in methods of determining the efficacy of a treatment for a GSD as well as methods of monitoring metabolic control status in a GSD subject and methods for determining the effect of regulating carbohydrate dietary intake.

1. A method of diagnosing a subject with a Glycogen Storage Disease (GSD), the method comprising:
obtaining a biosample from a subject suspected of having GSD I;
determining the level of 1,5-anhydroglucitol (1,5-AG) in the biosample; and
when the level of 1,5-AG in the biosample of the subject suspected of having a GSD is higher than that of a reference biosample, then diagnosing the subject with a GSD Ia or Ib.

2. The method of claim 1, wherein the level of 1,5-AG in the subject suspected of having a GSD is associated with a Z-score of at least +1.5, at least +2, or more than +2.

3. (canceled)

4. The method of claim 1, wherein the reference biosample comprises a biosample from a subject not having a GSD.

5. The method of claim 1, further comprising obtaining a reference biosample, wherein the biosample comprises blood, serum, or plasma.

6. The method of claim 5, wherein obtaining a reference biosample comprises obtaining a biosample from a subject not having a GSD and determining the level of 1,5-AG in the biosample.

7. The method of claim 1, wherein determining the level of 1,5-AG in the biosample comprises using mass spectrometry, ultra-performance liquid chromatography (UPLC), high-performance liquid chromatography (HPLC), mass spectrometry in conjunction with UPLC, LC/MS/MS, ELISA, Western blots, or any combination thereof.

8. (canceled)

9. The method of claim 1, further comprising determining the level of one or more cellular metabolites and comparing the level of the one or more cellular metabolites to that of the reference biosample to identify one or more differentially present cellular metabolites.

10. The method of claim 9, wherein the one or more differentially present cellular metabolites comprise serum cholinesterase, albumin, and any combination thereof, or wherein the one or more differentially present cellular metabolites comprise glucose, uric acid, lactate, triglycerides, and any combination thereof.

11. (canceled)

12. (canceled)

13. A method of determining the effectiveness of a treatment in a subject having a Glycogen Storage Disease (GSD), the method comprising:
administering a treatment to a subject having GSD I;
obtaining a post-treatment biosample from the subject and determining the level of 1,5-anhydroglucitol (1,5-AG) in the biosample; and
determining the effectiveness of the treatment by comparing the level of 1,5-AG in the post-treatment biosample to the level of 1,5-AG in either a pre-treatment biosample or a reference biosample.

14. The method of claim 13, further comprising obtaining a pre-treatment biosample from the subject and determining the level of 1,5-AG, wherein the biosample comprises blood, serum, or plasma.

15. The method of claim 13, further comprising obtaining a reference biosample from a subject not having a GSD and determining the level of 1,5-AG.

16. (canceled)

17. The method of claim 13, wherein the treatment comprises administering to the subject uncooked cornstarch, modified cornstarch, a cornstarch derivative, a cornstarch alternative, or a combination thereof.

18. The method of claim 13, wherein the treatment is effective
when the post-treatment level of 1,5-AG is lower than the pre-treatment level of 1,5-AG, or
when the post-treatment level of 1,5-AG is within an acceptable range of the reference level of 1,5-AG.

19. The method of claim 13, wherein when the treatment is effective, further comprising repeating the treatment.

20. The method of claim 13, wherein the treatment is not effective
when the post-treatment level of 1,5-AG is higher than the pre-treatment level of 1,5-AG, or
when the post-treatment level of 1,5-AG is not within an acceptable range of the reference level of 1,5-AG.

21. The method of claim 20, wherein when the treatment is not effective, then implementing a change in the subject's dietary intake of carbohydrates.

22. The method of claim 21, wherein implementing a change in the subject's dietary intake of carbohydrates comprises administering more cornstarch and/or carbohydrates to the subject, more frequently administering cornstarch and/or carbohydrates to the subject, or both.

23. The method of claim 13, wherein determining the level of 1,5-AG in a biosample comprises using mass spectrometry, ultra-performance liquid chromatography (UPLC), high-performance liquid chromatography (HPLC), mass spectrometry in conjunction with UPLC, LC/MS/MS, ELISA, Western blots, or any combination thereof.

24. (canceled)

25. The method of claim 13, further comprising determining the level of one or more cellular metabolites in the biosamples and comparing the level of the one or more cellular metabolites in the biosamples to identify one or more differentially present cellular metabolites.

26. The method of claim 25, wherein the one or more differentially present cellular metabolites comprise serum cholinesterase, albumin, and any combination thereof, or wherein the one or more differentially present cellular metabolites comprise glucose, uric acid, lactate, triglycerides, and any combination thereof.

27. (canceled)
